(19) United States
(12) Patent Application Publication
(10) Pub. No.: US 2026/0325432 A1
(43) Pub. Date: Oct. 22, 2026

(54) TASTE-MODIFYING COMPOSITIONS AND USES THEREOF

(71) Applicant: FIRMENICH SA, Satigny (CH)

(72) Inventors: Wolfgang FIEBER, Ornex (FR); Qing-Bo OUYANG, Plainsboro, NJ (US)

(73) Assignee: FIRMENICH SA, Satigny (CH)

(21) Appl. No.: 19/168,903

(22) PCT Filed: Mar. 26, 2024
(86) PCT No.: PCT/EP2024/058040

§ 371 Date: Sep. 25, 2025

Related U.S. Application Data
(60) Provisional application No. 63/492,535, filed on Mar. 28, 2023.

(30) Foreign Application Priority Data
Jul. 6, 2023 (EP) 23183763.4

(51) Int. Cl. A61K 8/92; A61Q 11/00

(52) U.S. Cl. CPC A61K 8/922; A61Q 11/00; A61K 2800/92

(57) ABSTRACT

Described herein are taste-modifying compositions and their use for reducing the unpleasant taste of a flavored product. The present disclosure further provides flavored products containing such taste-modifying compositions and one or more unpleasant tastants. The taste-modifying compositions described herein are suitable for use in food products, beverage products, or oral care products, such as toothpaste, dental gels, mouthwashes, mouth sprays, toothpaste, tablets and toothpaste powder.

### CROSS REFERENCE TO RELATED APPLICATIONS

This application claims the benefit of priority of U.S. Provisional Application No. 63/492,535, filed Mar. 28, 2023, and European Application No. 23183763.4, filed Jul. 6, 2023, which is hereby incorporated by reference as though set forth herein in its entirety.

### FIELD OF THE DISCLOSURE

The present disclosure generally provides taste-modifying compositions and their use for reducing the unpleasant taste of a flavored product. The present disclosure further provides flavored products comprising such taste-modifying compositions and an unpleasant tastant. The taste-modifying compositions described herein are suitable for use in food products, beverage products, or oral care products, such as toothpaste, dental gels, mouthwashes, mouth sprays, toothpaste tablets, toothpaste powder, and the like.

### BACKGROUND OF THE DISCLOSURE

The taste system provides sensory information about the chemical composition of the external world. Taste transduction is one of the more sophisticated forms of chemically triggered sensation in animals. Signaling of taste is found throughout the animal kingdom, from simple metazoans to the most complex of vertebrates. Mammals are believed to have five basic taste modalities: sweet, bitter, sour, salty, and umami.

Oral care products often contain many active ingredients, such as anti-bacterial, whitening, and anti-sensitivity ingredients, that result in off-notes in oral care products. The inclusion of such active ingredients can lead to off tastes that users find unpleasant. Limiting the concentration of such materials is a potential strategy for reducing the any unpleasant taste. However, it may often be desirable to include such ingredient at higher concentrations to have an improved effect.

Off-note masking agents exist to meet the needs of consumers to mask the off notes resulting from these ingredients. However, some off-note masking agents are not compatible, i.e., insoluble, with flavor oils typically used in food products, beverage products, or oral care products. This prevents them from being combined into a single flavor delivery system that can be conveniently handled and incorporated into a final product.

Therefore, there is an ongoing need for flavor delivery systems containing off-note masking agents and flavor oils that are easy-to-handle and stable (i.e., clear with no precipitation) for long periods of time at room temperature.

### SUMMARY OF THE DISCLOSURE

In a first aspect, the present disclosure relates to a taste-modifying composition comprising:
a) one or more flavor oils,
b) one or more off-note masking agents,
c) one or more non-polar surfactants,
d) one or more polar surfactants,
e) water, and
f) one or more water-miscible co-solvents.

In a second aspect, the present disclosure relates to a flavored product comprising the taste-modifying composition described herein and an unpleasant tastant.

In a third aspect, the present disclosure relates to a use of the taste-modifying composition described herein to reduce an unpleasant taste of a flavored product.

### DETAILED DESCRIPTION

The following detailed description sets forth various aspects and embodiments provided herein. The description is to be read from the perspective of the person of ordinary skill in the relevant art. Therefore, information that is well known to such ordinarily skilled artisans is not necessarily included. It would be apparent to ordinarily skilled artisans that the various aspects and embodiments provided herein may be combined in any manner without departing from the spirit of the disclosure.

The following terms and phrases have the meanings indicated below, unless otherwise provided herein. This disclosure may employ other terms and phrases not expressly defined herein. Such other terms and phrases have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary. Unless defined otherwise, all technical and scientific terms used herein have the same meaning as is commonly understood by one of skill in the art to which this specification pertains.

As used herein, the terms “a”, “an”, or “the” means “one or more” or “at least one” unless otherwise stated.

While compositions and methods are described in terms of “comprising,” “containing,” or “including” various components or steps, the compositions and methods can also “consist essentially of” or “consist of” the various components, substances and steps. As used herein the term “consisting essentially of” shall be construed to mean including the listed components, substances or steps and such additional components, substances or steps which do not materially affect the basic and novel properties of the composition or method. In some embodiments, a composition in accordance with embodiments of the present disclosure that “consists essentially of” the recited components or substances does not include any additional components or substances that alter the basic and novel properties of the composition.

It should be understood that any numerical range recited herein is intended to include all sub-ranges subsumed therein. For example, a range of “1 to 10” is intended to include all sub-ranges between and including the recited minimum value of 1 and the recited maximum value of 10; that is, having a minimum value equal to or greater than 1 and a maximum value of equal to or less than 10. Because the disclosed numerical ranges are continuous, they include every value between the minimum and maximum values. Unless expressly indicated otherwise, the various numerical ranges specified in this application are approximations.

As used herein, and unless otherwise indicated, the term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

As used herein, “optionally” means that the subsequently described event(s) may or may not occur. In some embodiments, the optional event does not occur. In some other embodiments, the optional event does occur one or more times.

Throughout the present disclosure, various publications may be incorporated by reference. Should the meaning of any language in such publications incorporated by reference conflict with the meaning of the language of the present disclosure, the meaning of the language of the present disclosure shall take precedence, unless otherwise indicated.

As used herein, “or” is to be given its broadest reasonable interpretation, and is not to be limited to an either/or construction. Thus, the phrase “comprising A or B” means that A can be present and not B, or that B is present and not A, or that A and B are both present. Further, if A, for example, defines a class that can have multiple members, e.g., A₁ and A₂, then one or more members of the class can be present concurrently.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure, and are not meant to be limiting in any fashion. Nor do these phrases indicate any kind of preference for the disclosed embodiment.

In the first aspect, the present disclosure relates to a taste-modifying composition comprising:
a) one or more flavor oils,
b) one or more off-note masking agents,
c) one or more non-polar surfactants,
d) one or more polar surfactants,
e) water, and
f) one or more water-miscible co-solvents.

The one or more flavor oils suitable for use in the taste-modifying composition described herein may be any flavor oil known to those of ordinary skill in the art. These flavor oils may be chosen from synthetic flavor oils and flavoring aromatics or oils, oleoresins and extracts derived from plants, leaves, flowers, fruits, and so forth, and combinations thereof. Non-limiting representative flavor oils include spearmint oil, cinnamon oil, oil of wintergreen (methyl salicylate), peppermint oil, Japanese mint oil, clove oil, bay oil, anise oil, eucalyptus oil, thyme oil, cedar leaf oil, oil of nutmeg, allspice, oil of sage, mace, oil of bitter almonds, cassia oil, and citrus oils, including, for example, lemon, orange, lime, grapefruit, yazu, sudachi, and fruit essences including apple, pear, peach, grape, blueberry, strawberry, raspberry, cherry, plum, pineapple, watermelon, apricot, banana, melon, apricot, ume, cherry, raspberry, blackberry, tropical fruit, mango, mangosteen, pomegranate, papaya and so forth.

In an embodiment, the one or more flavor oils is/are selected from the group consisting of mint oil, such as spearmint oil, peppermint oil, and Japanese mint oil; cinnamon oil, oil of wintergreen (methyl salicylate), clove oil, bay oil, anise oil, eucalyptus oil, thyme oil, cedar leaf oil, oil of nutmeg, allspice, oil of sage, mace, oil of bitter almonds, cassia oil, and citrus oil. In an embodiment, the one or more flavor oils comprises mint oil.

In some embodiments, the taste-modifying composition may comprise more than one flavor oil.

The amount of the one or more flavor oils present in the taste-modifying composition is not particularly limited. However, in some embodiments, the taste-modifying composition of the present disclosure allows for a significantly higher concentration of flavor oil. In an embodiment, the one or more flavor oils is/are present in an amount of from 20% to 60%, typically 40% to 55%, by weight relative to the total weight of the composition.

The one or more off-note masking agents suitable for use in the taste-modifying composition described herein may be any off-note masking agents known to those of ordinary skill in the art. Off-notes are undesirable taste notes that may be present in the taste-modifying composition or in a flavored product into which the taste-modifying composition may be incorporated. Exemplary off-notes include, but are not limited to, bitter notes, medicinal notes, metallic notes, or any combination thereof, and are typically due to the presence of unpleasant tastants. The perception of unpleasant tastes may be reduced using off-note masking agents, which are known to those of ordinary skill in the art. Exemplary off-note masking agents include, but are not limited to, glucosylated natural steviol glycosides.

As used herein, the term “natural steviol glycosides” refers to steviol glycosides naturally found in the plants of the species Stevia rebaudiana, Stevia phlebophylla, or Rubus chingii. Such compounds are glycosides of the diterpene, steviol. Some non-limiting examples include stevioside, rebaudioside A, rebaudioside C, dulcoside A, and the like. As used herein, the term “glucosylated natural steviol glycosides” refers to compounds obtained by the enzymatic glucosylation of natural steviol glycosides to increase the degree of glucosylation. As used herein, the term “GSG” is used interchangeably with “glucosylated natural steviol glycoside.”

Any suitable GSGs may be used in the various aspects and embodiments disclosed herein. Thus, the GSGs can have any suitable degree of additional glucosylation, and any suitable type of glucosylation. In some embodiments, the GSGs used in the taste-modifying composition have a degree of glucosylation of at least 1.0, meaning that at least one mole of glucose has been enzymatically added to the natural steviol glycosides per mole of natural steviol glycoside. In some other embodiments, the GSGs used in the taste-modifying composition have a degree of glucosylation of at least 1.1, or at least 1.2, or at least 1.3, or at least 1.4, or at least 1.5, or at least 1.6, or at least 1.7, or at least 1.8, or at least 1.9, or at least 2.0, or at least 2.1, or at least 2.2, or at least 2.3, or at least 2.4, or at least 2.5. The glucosylation can be of any suitable type. For example, in some embodiments, the glucose enzymatically added to the natural steviol glycosides is alpha-glucose, beta-glucose, or a combination thereof. In some embodiments, the glucose enzymatically added to the natural steviol glycosides is alpha-glucose. In some embodiments, the glucose enzymatically added to the natural steviol glycosides is beta-glucose. In some embodiments, the glucose enzymatically added to the natural steviol glycosides is a combination of alpha-glucose and beta-glucose. The glucose added by enzymatic glucosylation can be incorporated via any suitable connection. Typically, the added glucose units connect to other glucose glucose units already present on the natural steviol glycoside. In some embodiments, the enzymatically added glucose units are added via 1,6 linkages, meaning that the glucose units add via the 1-position of the added glucose unit and the 6-position of the glucose unit already present on the natural steviol glycoside. In some other embodiments, the enzymatically added glucose units are added via 1,4 linkages. In some other embodiments, the enzymatically added glucose units are added via 1,2 linkages.

In an embodiment, the one or more off-note masking agents comprise(s) one or more glucosylated natural steviol glycosides.

The amount of the one or more off-note masking agents in the taste-modifying composition according to the present disclosure is not particularly limited. However, in a suitable embodiment, the one or more off-note masking agents is/are present in an amount of from 0.01% to 5%, typically from 0.2% to 4%, by weight relative to the total weight of the composition.

The one or more non-polar surfactants suitable for use in the taste-modifying composition described herein may be any nonionic surfactant having a hydrophilic-lipophilic balance (HLB) value of equal to or less than 10.

As used herein, the hydrophilic-lipophilic balance (HLB) refers to the balance between the hydrophilic (water-loving) and lipophilic (oil-loving) properties of a surfactant molecule. Surfactants are molecules that have both hydrophilic and lipophilic moieties. The HLB value of a surfactant is a numerical value that indicates its relative hydrophilic and lipophilic character. A high HLB value (typically 10 and above) indicates a more hydrophilic surfactant, while a low HLB value (typically below 10) indicates a more lipophilic surfactant. The HLB value is often used to determine the efficacy of a surfactant in a particular application, such as emulsifying oils in water or water in oils, and therefore it is a practical guideline in formulation. HLB may also be used to describe the relationship between the molecular masses of hydrophilic and lipophilic moieties of an amphiphilic molecule (Griffin, W. C. Classification of surface-active agents by HLB. J. Soc. Cosmet. Chem. 1, 1949, 311-326).

Exemplary non-polar surfactants include, but are not limited to sorbitan fatty acid esters, typically selected from the group consisting of sorbitan monostearate (Span 60), sorbitan tristearate (Span 65), sorbitan monolaurate (Span 20), sorbitan monooleate (Span 80), sorbitan monopalmitate (Span 40), sorbitan trioleate (Span 85); lecithins, typically soy lecithin; (poly)glyceryl esters, typically glycerol monooleate; and mixtures thereof.

The amount of the one or more non-polar surfactants is not particularly limited. However, in a suitable embodiment, the one or more non-polar surfactants is/are present in an amount of from 1% to 40%, typically 5% to 35%, by weight relative to the total weight of the composition.

The one or more polar surfactants suitable for use in the taste-modifying compositions of the present disclosure may be any nonionic surfactant having a hydrophilic-lipophilic balance (HLB) value of greater than 10.

Exemplary polar surfactants include, but are not limited to, poly(oxyethylene)-poly(oxypropylene)-poly(oxyethylene) block copolymers, typically a triblock copolymer consisting of a central hydrophobic block of polypropylene glycol flanked by two hydrophilic blocks of polyethylene glycol (Poloxamer 407); one or more PEG sorbitan fatty acid esters, typically selected from the group consisting of PEG sorbitan monolaurate (Polysorbate 20), PEG sorbitan monooleate (Polysorbate 80), PEG sorbitan monopalmitate (Polysorbate 40), and PEG sorbitan stearate (Polysorbate 60); PEG-modified hydrogenated castor oil (Cremophor RH40); sugar esters, typically sucrose monopalmitate and sucrose monolaurate; (poly)glyceryl esters, such as decaglyceryl monooleate, alkyl glucosides, and mixtures thereof.

The amount of the one or more polar surfactants is not particularly limited. However, in a suitable embodiment, the one or more polar surfactants is/are present in an amount of from 1% to 40%, typically 5% to 35%, by weight relative to the total weight of the composition.

The one or more water-miscible co-solvents suitable for use in the taste-modifying composition of the present disclosure may be any solvent or blends of solvents that are miscible with water. Exemplary water-miscible co-solvents include, but are not limited to, propylene glycol, glycerol, ethanol, triethyl citrate, triacetin, ethyl lactate, ethyl acetate, propanol, isopropanol, and mixtures thereof.

In some instances, ethanol may not used in the taste-modifying composition of the present disclosure due to, for example, consumer demand for less astringent oral care products. Thus, in an embodiment, the the taste-modifying composition of the present disclosure is free of ethanol. As used herein, the phrase “free of” means that there is no external addition of the material to which it refers and that there is no detectable amount of the material that may be observed by analytical techniques known to the ordinarily skilled artisan, such as, for example, gas chromatography.

The amount of the one or more water-miscible co-solvents is not particularly limited. However, in a suitable embodiment, the one or more water-miscible co-solvents is/are present in an amount of from 0.01% to 30%, typically 5% to 25%, by weight relative to the total weight of the composition.

The taste-modifying composition of the present disclosure may be characterized by the weight ratio of the one or more flavor oils to the one or more off-note masking agents. In an embodiment, the weight ratio of the one or more flavor oils to the one or more off-note masking agents is in the range of from 20:1 to 100:1.

In some embodiments, the taste-modifying composition is in the form of a water-in-oil microemulsion.

As understood by a person of ordinary skill in the art, an emulsion, such as a macroemulsion, a nanoemulsion, or a microemulsion, is a mixture of two liquids that are immiscible due to their different polarities (hydrophobic vs. hydrophilic). In an emulsion, one liquid (dispersed, discontinuous, or internal phase) is dispersed in another liquid (external or continuous phase). One liquid is typically polar and the other liquid is typically non-polar. Therefore, the non-polar phase may be dispersed within the polar phase, or the polar phase may be dispersed within the non-polar phase. When the non-polar phase, typically an oil phase, is dispersed within the polar phase, typically an aqueous phase, such an emulsion is an oil-in-water emulsion. When the polar phase is dispersed within the non-polar phase, the emulsion is a water-in-oil emulsion.

Double emulsions, such as water-in-oil-in-water (WOW) emulsions and its opposite, oil-in-water-in-oil (OWO) emulsions, with three distinct phases are known. With respect to the WOW type, the three distinct phases consist of polar phase droplets that are dispersed in a non-polar phase, which is then enclosed in a continuous polar phase. Such three-phase emulsions are not contemplated in the present disclosure. Therefore, the emulsions of the present disclosure are not double emulsions, i.e., the emulsions of the present disclosure are neither water-in-oil-in-water (WOW) emulsions nor oil-in-water-in-oil (OWO) emulsions.

In an embodiment, the water-in-oil microemulsion comprises:
a′) a discontinuous aqueous phase comprising the one or more off-note masking agents and water; and
b′) a continuous oil phase comprising the one or more flavor oils.

The compositions according to the present disclosure are transparent at room temperature (RT) and are stable within a large temperature range (for example RT±10-20° C., typically between 10° C. and 45° C., more typically between 3° and 45° C.), and over an extended time period, typically a few days, more typically a few weeks, even more typically a few months or longer.

The term transparent means that the mixtures in the absence of coloring or fluorescent agents have transmittance values in the visible light (500-800 nm) of 100% at a path length of 1 cm referenced against demineralized water.

The term stable means that the mixtures do not phase separate and remain transparent without detectable signs of turbidity, cloudiness, or precipitates.

The taste-modifying compositions described herein may be prepared according to methods known to those of ordinary skill in the art. One suitable method comprises:
a′) preparing an oil phase by mixing one or more flavor oils, one or more non-polar surfactants, one or more polar surfactants, and one or more water-miscible solvents, and optionally heating the combination, typically at a temperature from 30° C. to 50° C., while stirring until a homogenous liquid is obtained;
b′) preparing an aqueous phase by mixing one or more off-note masking agents with water and stirring, typically at room temperature, until complete dissolution of the one or more off-note masking agents; and
c′) mixing the oil phase prepared in step a′) and the aqueous phase prepared in step
b′) while stirring, typically at room temperature, until a homogenous liquid, typically water-in-oil microemulsion, is obtained.

In the second aspect, the present disclosure relates to a flavored product comprising the taste-modifying composition described herein and an unpleasant tastant.

The amount of the taste-modifying composition in the flavored product is not particularly limited. In an embodiment, the taste-modifying composition is present in an amount of from 0.001% to 5%, typically 0.01% to 1%, more typically from 0.1% to 0.5%, by weight relative to the total weight of the flavored product.

In an embodiment, flavored product is a food product, a beverage product, an oral care product, or a pharmaceutical product.

In some embodiments, the flavored product further comprises a cooling compound, a cooling-enhancing compound, a sweetener other than steviol glycosides or mogrosides, or a combination thereof.

As used herein, a “cooling compound” refers to a compound that modulates the TRPM8 receptor.

Exemplary cooling compounds include, but are not limited to, compounds selected from the group consisting of: (−)-menthol, N-ethyl-p-menthyl-3-carboxamide, cubebol, isopulegol, 3-(L-menthoxy) propane-1,2-diol, cis/trans-p-menthane-3,8-diol, 3-(L-menthoxy)-2-methylpropane-1,2-diol, 2-[2-(p-menthan-3-yloxy) ethoxy]ethanol, menthoxyethanol, (1R,2R,4R)-1-(2-hydroxy-4-methylcyclohexyl) ethenone, (1R,2R,5R)—N-ethyl-5-methyl-2-(prop-1-en-2-yl)-cyclohexanecarboxamide, N-(3-hydroxy-4-methoxyphenyl)-2-isopropyl-5,5-dimethyl-cyclohexanecarboxamide, N-[4-(cyanomethyl)phenyl]-2-isopropyl-5,5-dimethyl-cyclohexanecarboxamide, N-(2-Hydroxy-2-phenylethyl)-2-isopropyl-5,5-dimethyl-cyclohexane-1-carboxamide, L-menthol lactate, (−)-menthyl ethylene glycol carbonate, (−)-menthone 1,2-glycerol ketal, D/L-menthone 1,2-glycerol ketal, (−)-menthyl 1-propylene glycol carbonate, (−)-menthyl 2-propylene glycol carbonate, D/L-menthyl 1-propylene glycol carbonate, D/L-menthyl 2-propylene glycol carbonate, (1R,2S,5R)—N-(4-methoxyphenyl)-5-methyl-2-(1-methylethyl)cyclohexanecarboxamide, menthyl ethylamido oxalate, (E)-3-benzo[1,3]dioxol-5-yl-N, N-diphenyl-2-propenamide, 3,4-methylenedioxycinnamic acid, N,N-diphenylamide, N,N-dimethyl (−)-menthyl succinimide, (1R,2S,5R)—N-(4-(carbamoylmethyl)phenyl)-menthylcarboxamide, (−)-menthyl pyrrolidone carboxylate, L-phenylephrine p-menthane carboxamide, (1R,2S,5R)—N-(4-(cyanomethyl)-phenyl)menthylcarboxamide, (1R,2S,5R)—N-(2-(pyridin-2-yl)ethyl)menthylcarboxamide, 6-isopropyl-3,9-dimethyl-1,4-dioxaspiro[4.5]decan-2-one, (1R,2R,4S)-dihydroumbellulol, N-ethyl-p-menthane-3-carboxamide, 2-isopropyl-N,2,3-trimethylbutyramide, ethyl 3-(p-menthane-3-carboxamido)acetate, N-ethyl-2,2-diisopropylbutanamide, N-(1,1-dimethyl-2-hydroxyethyl)-2,2-dimethylbutanamide, N-cyclopropyl-5-methyl-2-isopropyl-cyclohexanecarbonecarboxamide, (−)-menthyl acetoacetate, (−)-menthyl succinate, (−)-menthyl(S)-3-hydroxybutyrate, (−)-menthyl glutarate, 1-[2-hydroxyphenyl]-4-[2-nitrophenyl]-1,2,3,6-tetrahydropyrimidine-2-one, di-(−)-menthyl glutarate, N-(2-hydroxyethyl)-2,3-dimethyl-2-isopropylbutanamide, and any combinations thereof.

In some embodiments, the cooling compound may be a cooling compound set forth in WO 2021/074281, such as, for example, (2-(4-phenyl-1H-imidazol-2-yl)piperidin-1-yl)(1,2,3-thiadiazol-5-yl)methanone, (2-(4-phenyl-1H-imidazol-2-yl)piperidin-1-yl)(thietan-3-yl)methanone, (2,2-dimethylcyclopropyl)(2-(4-phenyl-1H-imidazol-2-yl)piperidin-1-yl)methanone, (2-ethoxycyclopropyl)(2-(4-phenyl-1H-imidazol-2-yl)piperidin-1-yl)methanone, (2-methoxycyclopropyl)(2-(4-phenyl-1H-imidazol-2-yl)piperidin-1-yl)methanone, (2-methylcyclopropyl)(2-(4-phenyl-1H-imidazol-2-yl)piperidin-1-yl)methanone, (2S)-2-methyl-1-(2-(5-(p-tolyl)-1H-imidazol-2-yl)piperidin-1-yl)butan-1-one, (5-methyltetrahydrofuran-3-yl)(2-(4-phenyl-1H-imidazol-2-yl)piperidin-1-yl)methanone, (E)-1-(2-(5-(3,4-difluorophenyl)-1H-imidazol-2-yl)piperidin-1-yl)-2-methylbut-2-en-1-one, (E)-2-methyl-1-(2-(5-(p-tolyl)-1H-imidazol-2-yl)piperidin-1-yl)but-2-en-1-one, (E)-2-methyl-1-(2-(5-(p-tolyl)-1H-imidazol-2-yl)piperidin-1-yl)pent-3-en-1-one, (R)-2-(methylthio)-1-((R)-2-(5-(p-tolyl)-1H-imidazol-2-yl)piperidin-1-yl)propan-1-one, (S)-2-(methylthio)-1-((R)-2-(5-(p-tolyl)-1H-imidazol-2-yl)piperidin-1-yl)propan-1-one, (S)-2-(methylthio)-1-((S)-2-(5-(p-tolyl)-1H-imidazol-2-yl)piperidin-1-yl)propan-1-one, (S)-2-methyl-1-((R)-2-(5-(p-tolyl)-1H-imidazol-2-yl)piperidin-1-yl)butan-1-one, (tetrahydrofuran-3-yl)(2-(5-(p-tolyl)-1H-imidazol-2-yl)piperidin-1-yl)methanone, 1-(2-(1H-benzo[d]imidazol-2-yl)piperidin-1-yl)-2-(methylthio)propan-1-one, 1-(2-(4-(1-methyl-1H-pyrazol-4-yl)-1H-imidazol-2-yl)piperidin-1-yl)-2-(methylthio)propan-1-one, 1-(2-(4-(2,2-difluorobenzo[d][1,3]dioxol-5-yl)-1H-imidazol-2-yl)piperidin-1-yl)-2-(methylthio)propan-1 one, 1-(2-(4-(2,3-dihydrobenzofuran-5-yl)-1H-imidazol-2-yl)piperidin-1-yl)-2-(methylthio)propan-1-one, 1-(2-(4-(2,3-dihydrobenzofuran-6-yl)-1H-imidazol-2-yl)piperidin-1-yl)-2-(methylthio)propan-1-one, 1-(2-(4-(2,5-dimethylphenyl)-1H-imidazol-2-yl)piperidin-1-yl)-2-(methylthio)propan-1-one, 1-(2-(4-(2-chlorophenyl)-1H-imidazol-2-yl)piperidin-1-yl)-2-(methylthio)propan-1-one, 1-(2-(4-(2-cyclopropyloxazol-5-yl)-1H-imidazol-2-yl)piperidin-1-yl)-2-(methylthio)propan-1-one, 1-(2-(4-(2-fluorophenyl)-1H-imidazol-2-yl)piperidin-1-yl)-2-(methylthio)propan-1-one, 1-(2-(4-(2-hydroxyphenyl)-1H-imidazol-2-yl)piperidin-1-yl)-2-(methylthio)propan-1-one, 1-(2-(4-(3-((dimethylamino)methyl)phenyl)-1H-imidazol-2-yl)piperidin-1-yl)-2-(methylthio)propan-1-one, 1-(2-(4-(3,5-difluorophenyl)-1H-imidazol-2-yl)piperidin-1-yl)-2-(methylthio)propan-1-one, 1-(2-(4-(3,5-dimethylisoxazol-4-yl)-1H-imidazol-2-yl)piperidin-1-yl)-2-(methylthio)propan-1-one, 1-(2-(4-(3-chloro-4-methylphenyl)-1H-imidazol-2-yl)piperidin-1-yl)-2-(methylthio)propan-1-one, 1-(2-(4-(3-methylfuran-2-yl)-1H-imidazol-2-yl)piperidin-1-yl)-2-(methylthio)propan-1-one, 1-(2-(4-(4-((1,1-dioxidothiomorpholino)methyl)phenyl)-1H-imidazol-2-yl)piperidin-1-yl)-2-(methylthio)propan-1-one, 1-(2-(4-(4-(1-hydroxyethyl)phenyl)-1H-imidazol-2-yl)piperidin-1-yl)-2-(methylthio)propan-1-one, 1-(2-(4-(4-(difluoromethoxy)-3-fluorophenyl)-1H-imidazol-2-yl)piperidin-1-yl)-2-(methylthio)propan-1-one, 1-(2-(4-(4-(difluoromethyl)thiophen-3-yl)-1H-imidazol-2-yl)piperidin-1-yl)-2-(methylthio)propan-1-one, 1-(2-(4-(4-(methylsulfonyl)phenyl)-1H-imidazol-2-yl)piperidin-1-yl)-2-(methylthio)propan-1-one, 1-(2-(4-(4-(tert-butyl)phenyl)-1H-imidazol-2-yl)piperidin-1-yl)-2-(methylthio)propan-1-one, 1-(2-(4-(4-cyclobutylphenyl)-1H-imidazol-2-yl)piperidin-1-yl)-2-(methylthio)propan-1-one, 1-(2-(4-(4-cyclopropyl-2-fluorophenyl)-1H-imidazol-2-yl)piperidin-1-yl)-2-(methylthio)propan-1-one, 1-(2-(4-(4-fluorophenyl)-1H-imidazol-2-yl)piperidin-1-yl)-2-(methylthio)propan-1-one, 1-(2-(4-(4-hydroxyphenyl)-1H-imidazol-2-yl)piperidin-1-yl)-2-(methylthio)propan-1-one, 1-(2-(4-(4-isopropylphenyl)-1H-imidazol-2-yl)piperidin-1-yl)-2-(methylthio)propan-1-one, 1-(2-(4-(4-methyl-3-(trifluoromethyl)phenyl)-1H-imidazol-2-yl)piperidin-1-yl)-2-(methylthio)propan-1-one, 1-(2-(4-(5-((dimethylamino)methyl)thiophen-2-yl)-1H-imidazol-2-yl)piperidin-1-yl)-2-(methylthio)propan-1-one, 1-(2-(4-(5-methylfuran-2-yl)-1H-imidazol-2-yl)piperidin-1-yl)-2-(methylthio)propan-1-one, 1-(2-(4-(5-methylpyridin-3-yl)-1H-imidazol-2-yl)piperidin-1-yl)-2-(methylthio)propan-1-one, 1-(2-(4-(6-methylpyridin-3-yl)-1H-imidazol-2-yl)piperidin-1-yl)-2-(methylthio)propan-1-one, 1-(2-(4-(benzo[b]thiophen-6-yl)-1H-imidazol-2-yl)piperidin-1-yl)-2-(methylthio)propan-1-one, 1-(2-(4-methyl-5-phenyl-1H-imidazol-2-yl)piperidin-1-yl)-2-(methylthio)propan-1-one, 1-(2-(4-phenyl-1H-imidazol-2-yl)piperidin-1-yl)-2-((trifluoromethyl)thio)ethan-1-one, 1-(2-(4-phenyl-1H-imidazol-2-yl)piperidin-1-yl)butan-1-one, 1-(2-(4-phenyl-1H-imidazol-2-yl)piperidin-1-yl) hexa-3,5-dien-1-one, 1-(2-(4-phenyl-1H-imidazol-2-yl)piperidin-1-yl)pent-4-en-1-one, 1-(2-(4-phenyl-1H-imidazol-2-yl)piperidin-1-yl)propan-1-one, 1-(2-(5-(2,3-difluorophenyl)-1H-imidazol-2-yl)piperidin-1-yl)-2-(methylthio)propan-1-one, 1-(2-(5-(2,4-difluorophenyl)-1H-imidazol-2-yl)piperidin-1-yl)-2-(methylthio)propan-1-one, 1-(2-(5-(2,5-difluorophenyl)-1H-imidazol-2-yl)piperidin-1-yl)-2-(methylthio)propan-1-one, 1-(2-(5-(2-fluoro-4-methoxyphenyl)-1H-imidazol-2-yl)piperidin-1-yl)-2-(methylthio)propan-1-one, 1-(2-(5-(2-fluoro-4-methylphenyl)-1H-imidazol-2-yl)piperidin-1-yl)-2-(methylthio)propan-1-one, 1-(2-(5-(2-methoxypyrimidin-5-yl)-1H-imidazol-2-yl)piperidin-1-yl)-2-(methylthio)propan-1-one, 1-(2-(5-(3-(methylamino)phenyl)-1H-imidazol-2-yl)piperidin-1-yl)-2-(methylthio)propan-1-one, 1-(2-(5-(3,4-difluorophenyl)-1H-imidazol-2-yl)piperidin-1-yl)-2-(methylthio)propan-1-one, 1-(2-(5-(3,4-difluorophenyl)-1H-imidazol-2-yl)piperidin-1-yl)-2-methylbut-3-en-1-one, 1-(2-(5-(3,4-dimethoxyphenyl)-1H-imidazol-2-yl)piperidin-1-yl)-2-(methylthio)propan-1-one, 1-(2-(5-(3,4-dimethylphenyl)-1H-imidazol-2-yl)piperidin-1-yl)-2-(methylthio)propan-1-one, 1-(2-(5-(3-fluoro-4-(methoxymethyl)phenyl)-1H-imidazol-2-yl)piperidin-1-yl)-2-(methylthio)propan-1-one, 1-(2-(5-(3-fluoro-4-methylphenyl)-1H-imidazol-2-yl)piperidin-1-yl)-2-(methylthio)propan-1-one, 1-(2-(5-(3-fluorophenyl)-1H-imidazol-2-yl)piperidin-1-yl)-2-(methylthio)propan-1-one, 1-(2-(5-(3-methoxyisothiazol-4-yl)-1H-imidazol-2-yl)piperidin-1-yl)-2-(methylthio)propan-1-one, 1-(2-(5-(3-methoxyphenyl)-1H-imidazol-2-yl)piperidin-1-yl)-2-(methylthio)propan-1-one, 1-(2-(5-(4-((methylamino)methyl)phenyl)-1H-imidazol-2-yl)piperidin-1-yl)-2-(methylthio)propan-1-one, 1-(2-(5-(4-(2-methoxyethyl)phenyl)-1H-imidazol-2-yl)piperidin-1-yl)-2-(methylthio)propan-1-one, 1-(2-(5-(4-(aminomethyl)phenyl)-1H-imidazol-2-yl)piperidin-1-yl)-2-(methylthio)propan-1-one, 1-(2-(5-(4-(dimethylamino)phenyl)-1H-imidazol-2-yl)piperidin-1-yl)-2-(methylthio)propan-1-one, 1-(2-(5-(4-(hydroxymethyl)phenyl)-1H-imidazol-2-yl)piperidin-1-yl)-2-(methylthio)propan-1-one, 1-(2-(5-(4-acetylphenyl)-1H-imidazol-2-yl)piperidin-1-yl)-2-(methylthio)propan-1-one, 1-(2-(5-(4-aminophenyl)-1H-imidazol-2-yl)piperidin-1-yl)-2-(methylthio)propan-1-one, 1-(2-(5-(4-chloro-3-fluorophenyl)-1H-imidazol-2-yl)piperidin-1-yl)-2-(methylthio)propan-1-one, 1-(2-(5-(4-chlorophenyl)-1H-imidazol-2-yl)piperidin-1-yl)-2-(methylthio)propan-1-one, 1-(2-(5-(4-ethylphenyl)-1H-imidazol-2-yl)piperidin-1-yl)-2-(methylthio)propan-1-one, 1-(2-(5-(4-fluorophenyl)-4H-1,2,4-triazol-3-yl)piperidin-1-yl)-2-(methylthio)propan-1-one, 1-(2-(5-(4-methoxy-2-methylphenyl)-1H-imidazol-2-yl)piperidin-1-yl)-2-(methylthio)propan-1-one, 1-(2-(5-(4-methoxyphenyl)-1H-imidazol-2-yl)piperidin-1-yl)-2-(methylthio)propan-1-one, 1-(2-(5-(4-methyl-6-(methylthio)pyridin-3-yl)-1H-imidazol-2-yl)piperidin-1-yl)-2-(methylthio)propan-1-one, 1-(2-(5-(5-(methoxymethyl)thiophen-3-yl)-1H-imidazol-2-yl)piperidin-1-yl)-2-(methylthio)propan-1-one, 1-(2-(5-(6-methoxypyridin-3-yl)-1H-imidazol-2-yl)piperidin-1-yl)-2-(methylthio)propan-1-one, 1-(2-(5-(benzo[d][1,3]dioxol-5-yl)-1H-imidazol-2-yl)piperidin-1-yl)-2-(methylthio)propan-1-one, 1-(2-(5-(p-tolyl)-1H-imidazol-2-yl)piperidin-1-yl)propan-1-one, 1-(2-(5-benzyl-1H-imidazol-2-yl)piperidin-1-yl)-2-(methylthio)propan-1-one, 1-(2-(5-bromo-1H-imidazol-2-yl)piperidin-1-yl)-2-(methylthio)propan-1-one, 2-(2-(1-(2-(methylthio)propanoyl)piperidin-2-yl)-1H-imidazol-5-yl)benzonitrile, 2-(2-(4-phenyl-1H-imidazol-2-yl)piperidine-1-carbonyl)cyclopropane-1-carbonitrile, 2-(5-methylfuran-2-yl)-1-(2-(4-phenyl-1H-imidazol-2-yl)piperidin-1-yl)propan-1-one, 2-(allylthio)-1-(2-(5-(p-tolyl)-1H-imidazol-2-yl)piperidin-1-yl)propan-1-one, 2-(dimethylamino)-1-(2-(4-phenyl-1H-imidazol-2-yl)piperidin-1-yl)butan-1-one, 2-(dimethylamino)-1-(2-(4-phenyl-1H-imidazol-2-yl)piperidin-1-yl)propan-1-one, 2-(ethylthio)-1-(2-(5-(p-tolyl)-1H-imidazol-2-yl)piperidin-1-yl)propan-1-one, 2-(furan-2-yl)-1-(2-(4-phenyl-1H-imidazol-2-yl)piperidin-1-yl) ethan-1-one, 2-(furan-2-yl)-1-(2-(4-phenyl-1H-imidazol-2-yl)piperidin-1-yl)propan-1-one, 2-(furan-3-yl)-1-(2-(4-phenyl-1H-imidazol-2-yl)piperidin-1-yl) ethan-1-one, 2-(furan-3-yl)-1-(2-(5-(p-tolyl)-1H-imidazol-2-yl)piperidin-1-yl)propan-1-one, 2-(methylamino)-N-(4-(2-(1-(2-(methylthio)propanoyl)piperidin-2-yl)-1H-imidazol-5-yl)phenyl)acetamide, 2-(methylsulfinyl)-1-(2-(5-(p-tolyl)-1H-imidazol-2-yl)piperidin-1-yl)propan-1-one, 2-(methylsulfonyl)-1-(2-(4-phenyl-1H-imidazol-2-yl)piperidin-1-yl) ethan-1-one, 2-(methylsulfonyl)-1-(2-(4-phenyl-1H-imidazol-2-yl)piperidin-1-yl)propan-1-one, 2-(methylthio)-1-(2-(2-(p-tolyl)-2H-tetrazol-5-yl)piperidin-1-yl)propan-1-one, 2-(methylthio)-1-(2-(3-phenyl-1,2,4-oxadiazol-5-yl)piperidin-1-yl)propan-1-one, 2-(methylthio)-1-(2-(4-(1-(trifluoromethyl)-1H-pyrazol-4-yl)-1H-imidazol-2-yl)piperidin-1-yl)propan-1-one, 2-(methylthio)-1-(2-(4-(4-(morpholinomethyl)phenyl)-1H-imidazol-2-yl)piperidin-1-yl)propan-1-one, 2-(methylthio)-1-(2-(4-(5-(trifluoromethyl) furan-2-yl)-1H-imidazol-2-yl)piperidin-1-yl)propan-1-one, 2-(methylthio)-1-(2-(4-(5-(trifluoromethyl)thiophen-2-yl)-1H-imidazol-2-yl)piperidin-1-yl)propan-1-one, 2-(methylthio)-1-(2-(4-(5-methylthiophen-3-yl)-1H-imidazol-2-yl)piperidin-1-yl)propan-1-one, 2-(methylthio)-1-(2-(4-(m-tolyl)-1H-imidazol-2-yl)piperidin-1-yl)propan-1-one, 2-(methylthio)-1-(2-(4-(o-tolyl)-1H-imidazol-2-yl)piperidin-1-yl)propan-1-one, 2-(methylthio)-1-(2-(4-(pyridin-3-yl)-1H-imidazol-2-yl)piperidin-1-yl)propan-1-one, 2-(methylthio)-1-(2-(4-(pyridin-4-yl)-1H-imidazol-2-yl)piperidin-1-yl)propan-1-one, 2-(methylthio)-1-(2-(4-(pyrimidin-5-yl)-1H-imidazol-2-yl)piperidin-1-yl)propan-1-one, 2-(methylthio)-1-(2-(4-(quinoxalin-6-yl)-1H-imidazol-2-yl)piperidin-1-yl)propan-1-one, 2-(methylthio)-1-(2-(4-(thiophen-3-yl)-1H-imidazol-2-yl)piperidin-1-yl)propan-1-one, 2-(methylthio)-1-(2-(4-phenyl-1H-imidazol-2-yl)piperidin-1-yl) ethan-1-one, 2-(methylthio)-1-(2-(5-(2-(trifluoromethyl)phenyl)-1H-imidazol-2-yl)piperidin-1-yl)propan-1-one, 2-(methylthio)-1-(2-(5-(2,3,4-trifluorophenyl)-1H-imidazol-2-yl)piperidin-1-yl)propan-1-one, 2-(methylthio)-1-(2-(5-(2,4,5-trifluorophenyl)-1H-imidazol-2-yl)piperidin-1-yl)propan-1-one, 2-(methylthio)-1-(2-(5-(3-(trifluoromethyl)phenyl)-1H-imidazol-2-yl)piperidin-1-yl)propan-1-one, 2-(methylthio)-1-(2-(5-(4-(trifluoromethoxy)phenyl)-1H-imidazol-2-yl)piperidin-1-yl)propan-1-one, 2-(methylthio)-1-(2-(5-(4-(trifluoromethyl)phenyl)-1H-imidazol-2-yl)piperidin-1-yl)propan-1-one, 2-(methylthio)-1-(2-(5-(4-propylphenyl)-1H-imidazol-2-yl)piperidin-1-yl)propan-1-one, 2-(methylthio)-1-(2-(5-(4-vinylphenyl)-1H-imidazol-2-yl)piperidin-1-yl)propan-1-one, 2-(methylthio)-1-(2-(5-(5-methylthiophen-2-yl)-1H-imidazol-2-yl)piperidin-1-yl)propan-1-one, 2-(methylthio)-1-(2-(5-(p-tolyl)-1,3,4-oxadiazol-2-yl)piperidin-1-yl)propan-1-one, 2-(methylthio)-1-(2-(5-(p-tolyl)-1H-imidazol-2-yl) azepan-1-yl)propan-1-one, 2-(methylthio)-1-(2-(5-(p-tolyl)-1H-imidazol-2-yl)piperidin-1-yl)propan-1-one, 2-(methylthio)-1-(2-(5-(p-tolyl)-4H-1,2,4-triazol-3-yl)piperidin-1-yl)propan-1-one, 2-(methylthio)-1-(2-(5-(p-tolyl) isoxazol-3-yl)piperidin-1-yl)propan-1-one, 2-(methylthio)-1-(2-(5-(p-tolyl) oxazol-2-yl)piperidin-1-yl)propan-1-one, 2-(methylthio)-1-(2-(5-(p-tolyl) thiazol-2-yl)piperidin-1-yl)propan-1-one, 2-(methylthio)-1-(2-(5-phenyl-1H-imidazol-2-yl)piperidin-1-yl)propan-1-one, 2-(methylthio)-1-(2-(5-phenyl-1H-imidazol-2-yl) pyrrolidin-1-yl)propan-1-one, 2-(methylthio)-1-(2-(5-phenyl-4H-1,2,4-triazol-3-yl)piperidin-1-yl)propan-1-one, 2-(methylthio)-1-(2-(5-phenyloxazol-2-yl)piperidin-1-yl)propan-1-one, 2-(methylthio)-1-(4-(5-(p-tolyl)-1H-imidazol-2-yl) thiazolidin-3-yl)propan-1-one, 2,2,3-trimethyl-1-(2-(5-(p-tolyl)-1H-imidazol-2-yl)piperidin-1-yl)but-3-en-1-one, 2,2,3-trimethyl-1-(2-(5-(p-tolyl)-1H-imidazol-2-yl)piperidin-1-yl)butan-1-one, 2,2-dimethyl-1-(2-(5-(p-tolyl)-1H-imidazol-2-yl)piperidin-1-yl)but-3-en-1-one, 2,2-dimethyl-1-(2-(5-(p-tolyl)-1H-imidazol-2-yl)piperidin-1-yl)butan-1-one, 2,3-dimethoxy-1-(2-(4-phenyl-1H-imidazol-2-yl)piperidin-1-yl)propan-1-one, 2,3-dimethyl-1-(2-(4-phenyl-1H-imidazol-2-yl)piperidin-1-yl)butan-1-one, 2,5-difluoro-4-(2-(1-(2-(methylthio)propanoyl)piperidin-2-yl)-1H-imidazol-4-yl)benzonitrile, 2-amino-1-(2-(5-(p-tolyl)-1H-imidazol-2-yl)piperidin-1-yl)propan-1-one, 2-cyclobutyl-1-(2-(4-phenyl-1H-imidazol-2-yl)piperidin-1-yl)propan-1-one, 2-cyclopentyl-2-ethoxy-1-(2-(4-phenyl-1H-imidazol-2-yl)piperidin-1-yl) ethan-1-one, 2-cyclopropyl-1-(2-(4-phenyl-1H-imidazol-2-yl)piperidin-1-yl) ethan-1-one, 2-cyclopropyl-1-(2-(4-phenyl-1H-imidazol-2-yl)piperidin-1-yl)propan-1-one, 2-cyclopropyl-2-methoxy-1-(2-(4-phenyl-1H-imidazol-2-yl)piperidin-1-yl) ethan-1-one, 2-ethoxy-1-(2-(5-(3-fluorophenyl)-1H-imidazol-2-yl)piperidin-1-yl)propan-1-one, 2-ethyl-1-(2-(4-phenyl-1H-imidazol-2-yl)piperidin-1-yl)but-2-en-1-one, 2-ethyl-1-(2-(4-phenyl-1H-imidazol-2-yl)piperidin-1-yl)butan-1-one, 2-fluoro-4-(2-(1-(2-(methylthio)propanoyl)piperidin-2-yl)-1H-imidazol-5-yl)benzonitrile, 2-mercapto-1-(2-(5-(p-tolyl)-1H-imidazol-2-yl)piperidin-1-yl)propan-1-one, 2-methoxy-1-(2-(4-phenyl-1H-imidazol-2-yl)piperidin-1-yl)butan-1-one, 2-methoxy-1-(2-(4-phenyl-1H-imidazol-2-yl)piperidin-1-yl)propan-1-one, 2-methoxy-1-(2-(5-(p-tolyl)-1H-imidazol-2-yl)piperidin-1-yl)propan-1-one, 2-methyl-1-(2-(4-phenyl-1H-imidazol-2-yl)piperidin-1-yl)butan-1-one, 2-methyl-1-(2-(4-phenyl-1H-imidazol-2-yl)piperidin-1-yl)propan-1-one, 2-methyl-1-(2-(5-(p-tolyl)-1H-imidazol-2-yl)piperidin-1-yl)but-3-en-1-one, 2-methyl-1-(2-(5-(p-tolyl)-1H-imidazol-2-yl)piperidin-1-yl)butan-1-one, 2-methyl-1-(2-(5-(p-tolyl)-1H-imidazol-2-yl)piperidin-1-yl)butane-1,3-dione, 2-methyl-1-(2-(5-(p-tolyl)-1H-imidazol-2-yl)piperidin-1-yl)pentan-1-one, 2-methyl-1-(2-(5-(p-tolyl)-1H-imidazol-2-yl)piperidin-1-yl)prop-2-en-1-one, 2-methyl-1-(2-(5-(p-tolyl)-1H-imidazol-2-yl)piperidin-1-yl)propan-1-one, 2-methyl-2-(methylthio)-1-(2-(5-(p-tolyl)-1H-imidazol-2-yl)piperidin-1-yl)propan-1-one, 2-methyl-3-(methylthio)-1-(2-(4-phenyl-1H-imidazol-2-yl)piperidin-1-yl)propan-1-one, 2-methylene-1-(2-(5-(p-tolyl)-1H-imidazol-2-yl)piperidin-1-yl)butan-1-one, 3-(2-(1-(2-(methylthio)propanoyl)piperidin-2-yl)-1H-imidazol-4-yl)benzonitrile, 3,3,3-trifluoro-2-methyl-1-(2-(4-phenyl-1H-imidazol-2-yl)piperidin-1-yl)propan-1-one, 3,3,4,4,4-pentafluoro-1-(2-(4-phenyl-1H-imidazol-2-yl)piperidin-1-yl)butan-1-one, 3,3-difluoro-1-(2-(4-phenyl-1H-imidazol-2-yl)piperidin-1-yl)butan-1-one, 3-fluoro-4-(2-(1-(2-(methylthio)propanoyl)piperidin-2-yl)-1H-imidazol-5-yl)benzonitrile, 3-methyl-1-(2-(4-phenyl-1H-imidazol-2-yl)piperidin-1-yl)but-2-en-1-one, 3-methyl-1-(2-(4-phenyl-1H-imidazol-2-yl)piperidin-1-yl)butan-1-one, 4-(2-(1-(2-(methylthio)propanoyl)piperidin-2-yl)-1H-imidazol-4-yl)benzonitrile, 4-(2-(1-(2-(methylthio)propanoyl)piperidin-2-yl)-1H-imidazol-5-yl)benzaldehyde, 4,4,4-trifluoro-2-methyl-1-(2-(4-phenyl-1H-imidazol-2-yl)piperidin-1-yl)butan-1-one, bicyclo[1.1.1]pentan-1-yl(2-(4-phenyl-1H-imidazol-2-yl)piperidin-1-yl)methanone, cyclobutyl(2-(5-(3-fluorophenyl)-1H-imidazol-2-yl)piperidin-1-yl)methanone, cyclopentyl(2-(4-phenyl-1H-imidazol-2-yl)piperidin-1-yl)methanone, cyclopropyl(2-(4-phenyl-1H-imidazol-2-yl)piperidin-1-yl)methanone, methyl 4-(2-(1-(2-(methylthio)propanoyl)piperidin-2-yl)-1H-imidazol-5-yl)benzoate, N-(2-hydroxyethyl)-4-(2-(1-(2-(methylthio)propanoyl)piperidin-2-yl)-1H-imidazol-4-yl)benzenesulfonamide, 2-(methylthio)-1-(2-(5-(3-nitrophenyl)-1H-imidazol-2-yl)piperidin-1-yl)propan-1-one, N-(4-(2-(1-(2-(methylthio)propanoyl)piperidin-2-yl)-1H-imidazol-4-yl)phenyl)methane-sulfonamide, 2-(methylthio)-1-(2-(5-(4-nitrophenyl)-1H-imidazol-2-yl)piperidin-1-yl)propan-1-one, 1-(sec-butylsulfinyl)-2-(5-(p-tolyl)-1H-imidazol-2-yl)piperidine, 1-(sec-butylsulfonyl)-2-(5-(p-tolyl)-1H-imidazol-2-yl)piperidine, 1-(isopropylsulfinyl)-2-(5-(p-tolyl)-1H-imidazol-2-yl)piperidine, 1-(isopropylsulfonyl)-2-(5-(p-tolyl)-1H-imidazol-2-yl)piperidine, 1-((1-(methylthio)ethyl)sulfinyl)-2-(5-(p-tolyl)-1H-imidazol-2-yl)piperidine, 1-((1-(methylthio)ethyl)sulfonyl)-2-(5-(p-tolyl)-1H-imidazol-2-yl)piperidine, 1-((2-(methylthio)propan-2-yl)sulfinyl)-2-(5-(p-tolyl)-1H-imidazol-2-yl)piperidine, 1-((2-(methylthio)propan-2-yl)sulfonyl)-2-(5-(p-tolyl)-1H-imidazol-2-yl)piperidine, 1-(but-3-en-2-ylsulfinyl)-2-(5-(p-tolyl)-1H-imidazol-2-yl)piperidine, 1-(but-3-en-2-ylsulfonyl)-2-(5-(p-tolyl)-1H-imidazol-2-yl)piperidine, 1-(pent-3-en-2-ylsulfinyl)-2-(5-(p-tolyl)-1H-imidazol-2-yl)piperidine, 1-(pent-3-en-2-ylsulfonyl)-2-(5-(p-tolyl)-1H-imidazol-2-yl)piperidine, 1-((2-methylbut-3-en-2-yl)sulfinyl)-2-(5-(p-tolyl)-1H-imidazol-2-yl)piperidine, 1-((2-methylbut-3-en-2-yl)sulfonyl)-2-(5-(p-tolyl)-1H-imidazol-2-yl)piperidine, 1-(2-(5-(4-(hydroxymethyl)-phenyl)-1H-imidazol-2-yl)piperidin-1-yl)-2-methylbutan-1-one, 2-(methylsulfonyl)-1-(2-(5-(p-tolyl)-1H-imidazol-2-yl)piperidin-1-yl)propan-1-one, 1-(4-hydroxy-2-(5-(p-tolyl)-1H-imidazol-2-yl)piperidin-1-yl)-2-(methylthio)propan-1-one, 1-(5-hydroxy-2-(5-(p-tolyl)-1H-imidazol-2-yl)piperidin-1-yl)-2-(methylthio)propan-1-one, 1-(4-hydroxy-2-(5-(p-tolyl)-1H-imidazol-2-yl)piperidin-1-yl)-2-methylbutan-1-one, 1-(5-hydroxy-2-(5-(p-tolyl)-1H-imidazol-2-yl)piperidin-1-yl)-2-methylbutan-1-one, 1-(2-(5-(4-(hydroxymethyl)phenyl)-1H-imidazol-2-yl)piperidin-1-yl)-2-(methylsulfonyl)propan-1-one, 1-(4-hydroxy-2-(5-(p-tolyl)-1H-imidazol-2-yl)piperidin-1-yl)-2-(methylsulfonyl)propan-1-one, 1-(5-hydroxy-2-(5-(p-tolyl)-1H-imidazol-2-yl)piperidin-1-yl)-2-(methylsulfonyl)propan-1-one, 1-(2-hydroxy-6-(5-(p-tolyl)-1H-imidazol-2-yl)piperidin-1-yl)-2-(methylthio)propan-1-one, 1-(2-hydroxy-6-(5-(p-tolyl)-1H-imidazol-2-yl)piperidin-1-yl)-2-methylbutan-1-one, 1-(2-hydroxy-6-(5-(p-tolyl)-1H-imidazol-2-yl)piperidin-1-yl)-2-(methylsulfonyl)propan-1-one, 1-(3-hydroxy-2-(5-(p-tolyl)-1H-imidazol-2-yl)piperidin-1-yl)-2-(methylthio)propan-1-one, 1-(3-hydroxy-2-(5-(p-tolyl)-1H-imidazol-2-yl)piperidin-1-yl)-2-methylbutan-1-one, 1-(3-hydroxy-2-(5-(p-tolyl)-1H-imidazol-2-yl)piperidin-1-yl)-2-(methylsulfonyl)propan-1-one, 1-(2-(5-(4-(hydroxymethyl)phenyl)-1H-imidazol-2-yl)piperidin-1-yl)-2-(methylsulfinyl)-propan-1-one, 1-(4-hydroxy-2-(5-(p-tolyl)-1H-imidazol-2-yl)piperidin-1-yl)-2-(methylsulfinyl)propan-1-one, 1-(5-hydroxy-2-(5-(p-tolyl)-1H-imidazol-2-yl)piperidin-1-yl)-2-(methylsulfinyl)propan-1-one, 1-(2-hydroxy-6-(5-(p-tolyl)-1H-imidazol-2-yl)piperidin-1-yl)-2-(methylsulfinyl)propan-1-one, 1-(3-hydroxy-2-(5-(p-tolyl)-1H-imidazol-2-yl)piperidin-1-yl)-2-(methylsulfinyl)propan-1-one, 1-(2-hydroxy-6-(5-(4-(hydroxymethyl)phenyl)-1H-imidazol-2-yl)piperidin-1-yl)-2-(methylthio)propan-1-one, 1-(5-hydroxy-2-(5-(4-(hydroxymethyl)phenyl)-1H-imidazol-2-yl)piperidin-1-yl)-2-(methylthio)propan-1-one, 1-(4-hydroxy-2-(5-(4-(hydroxymethyl)phenyl)-1H-imidazol-2-yl)piperidin-1-yl)-2-(methylthio)propan-1-one, 1-(3-hydroxy-2-(5-(4-(hydroxymethyl)-phenyl)-1H-imidazol-2-yl)piperidin-1-yl)-2-(methylthio)propan-1-one, 1-(2-hydroxy-6-(5-(4-(hydroxymethyl)phenyl)-1H-imidazol-2-yl)piperidin-1-yl)-2-methylbutan-1-one, 1-(5-hydroxy-2-(5-(4-(hydroxymethyl)phenyl)-1H-imidazol-2-yl)piperidin-1-yl)-2-methylbutan-1-one, 1-(4-hydroxy-2-(5-(4-(hydroxymethyl)phenyl)-1H-imidazol-2-yl)piperidin-1-yl)-2-methylbutan-1-one, 1-(3-hydroxy-2-(5-(4-(hydroxymethyl)phenyl)-1H-imidazol-2-yl)piperidin-1-yl)-2-methylbutan-1-one, 4-(2-(1-(2-methylbutanoyl)piperidin-2-yl)-1H-imidazol-5-yl)benzaldehyde, 4-(2-(1-(2-(methylthio)propanoyl)piperidin-2-yl)-1H-imidazol-5-yl)benzoic acid, 4-(2-(1-(2-methylbutanoyl)piperidin-2-yl)-1H-imidazol-5-yl)benzoic acid, (2,2-dimethylcyclopropyl)(2-(5-(p-tolyl)-1H-imidazol-2-yl)piperidin-1-yl)methanone, 1-(2-(5-bromo-1H-imidazol-2-yl)piperidin-1-yl)-2-methylbutan-1-one, 1-(2-(5-(4-chlorophenyl)-1H-imidazol-2-yl)piperidin-1-yl)-2-methylbutan-1-one, 2-(4-(2-(1-(2-(methylthio)propanoyl)piperidin-2-yl)-1H-imidazol-5-yl)phenyl)acetonitrile, 2-(4-(2-(1-(2-methylbutanoyl)piperidin-2-yl)-1H-imidazol-5-yl)phenyl)acetonitrile, 2-(4-(2-(1-(2-methylbutanoyl)piperidin-2-yl)-1H-imidazol-5-yl)phenyl)acetonitrile, 2,3-dimethyl-1-(2-(5-(p-tolyl)-1H-imidazol-2-yl)piperidin-1-yl)butan-1-one, 2-methyl-1-(2-(5-(o-tolyl)-1H-imidazol-2-yl)piperidin-1-yl)butan-1-one, 2-methyl-1-(2-(5-(m-tolyl)-1H-imidazol-2-yl)piperidin-1-yl)butan-1-one, 1-(2-(5-(4-(hydroxymethyl)phenyl)-1H-imidazol-2-yl)piperidin-1-yl)-2-methylbutan-1-one, 1-(isopropylsulfonyl)-2-(5-(p-tolyl)-1H-imidazol-2-yl)piperidine, 1-(sec-butylsulfonyl)-2-(5-(p-tolyl)-1H-imidazol-2-yl)piperidine, 2-methyl-1-(2-(5-(5-methylthiophen-2-yl)-1H-imidazol-2-yl)piperidin-1-yl)butan-1-one, 2-(methylthio)-1-(2-(5-(p-tolyl)-1H-imidazol-2-yl)piperazin-1-yl)propan-1-one, 2-(methylthio)-1-(2-(4-(p-tolyl) oxazol-2-yl)piperidin-1-yl)propan-1-one, 2-methyl-1-(2-(4-(p-tolyl) oxazol-2-yl)piperidin-1-yl)butan-1-one, derivatives thereof, such as those set forth in WO 2022/170519, or any combination thereof.

In some embodiments, the cooling compound may be 2-methyl-1-(2-(5-(p-tolyl)-1H-imidazol-2-yl)piperidin-1-yl)butan-1-one, 2-(methylthio)-1-(2-(5-(p-tolyl)-1H-imidazol-2-yl)piperidin-1-yl)propan-1-one, 2-methyl-2-(methylthio)-1-(2-(5-(p-tolyl)-1H-imidazol-2-yl)piperidin-1-yl)propan-1-one, 2,2-dimethyl-1-(2-(5-(p-tolyl)-1H-imidazol-2-yl)piperidin-1-yl)but-3-en-1-one, or a combination thereof.

In some embodiments, the cooling compound may be 1-isopropyl-3,3,5,7-tetramethyloctahydrobenzo[c]isoxazole, 5-(4-fluorophenyl)-1,3,3,5,7-pentamethyloctahydrobenzo[c]isoxazole (including (3aR,5R,7S,7aR)-5-(4-fluorophenyl)-1,3,3,5,7-pentamethyloctahydrobenzo[c]isoxazole, (3aR,5R,7R,7aR)-5-(4-fluorophenyl)-1,3,3,5,7-pentamethyloctahydrobenzo[c]isoxazole, (3aR,5R,7S,7aS)-5-(4-fluorophenyl)-1,3,3,5,7-pentamethyloctahydrobenzo[c]isoxazole, or any combination thereof.

As used herein, a “cooling-enhancing compound” refers to a compound capable of enhancing the cooling sensation provided by a flavored product having a cooling compound but may also provide a cooling sensation alone. Such cooling enhancing compounds include, but are not limited to, naturally derived compounds, such as menthol or analogs thereof, or synthetic compounds, such as any compounds set forth in U.S. Pat. Nos. 9,394,287 and 10,421,727. Exemplary cooling-enhancing compounds include, but are not limited to, compounds selected from the group consisting of N-ethyl-N-(thiophen-2-ylmethyl)-2-(p-tolyloxy)acetamide; N-(1H-pyrazol-3-yl)-N-(thiophen-2-ylmethyl)-2-(p-tolyloxy)acetamide; 2-(4-fluorophenoxy)-N-(1H-pyrazol-3-yl)-N-(thiophen-2-ylmethyl)acetamide; 2-(2-hydroxy-4-methylphenoxy)-N-(1H-pyrazol-3-yl)-N-(thiophen-2-ylmethyl)-acetamide; 2-((2,3-dihydro-1H-inden-5-yl)oxy)-N-(1H-pyrazol-3-yl)-N-(thiophen-2-ylmethyl)-acetamide; 2-((2,3-dihydro-1H-inden-5-yl)oxy)-N-(1H-pyrazol-3-yl)-N-(thiazol-5-ylmethyl)-acetamide; 5-methoxy-N-(1H-pyrazol-3-yl)-N-(thiophen-2-ylmethyl)benzofuran-2-carboxamide; 2-(4-ethylphenoxy)-N-(1H-pyrazol-3-yl)-N-(thiophen-2-ylmethyl)acetamide; (E/Z)-2-methyl-2-butenal; (E/Z)-2-isopropyl-5-methyl-2-hexenal; derivatives thereof, such as those set forth in WO 2022/170519, and any combinations thereof.

The flavored product may further comprise a sweetener. The sweetener may comprise a low-calorie, zero-calorie, or high-intensity sweetener. The term “high-intensity sweetener” refers to sweeteners that are sweeter than sucrose, such as at least 10 times, or at least 20 times, or at least 50 times, or at least 100 times, or at least 250 times, or at least 500 times, or at least 750 times, or at least 1000 times, or at least 2500 times, or at least 5000 times, or at least 7500 times, or at least 10000 times, sweeter than sucrose. In this context, to say that a sweetener is x times sweeter than sucrose means that a concentration of 1/x by weight relative to sucrose in aqueous solution of said sweetener yields an equivalent sweetness as sucrose.

Examples of suitable sweeteners are set forth in greater detail below. In some embodiments, the sweetener is sucralose, which refers to 1,6-dichloro-1,6-dideoxy-β-D-fructofuranosyl-4-chloro-4-deoxy-α-D-galactopyranoside. In some embodiments, the sweetener is sodium saccharin. The sodium saccharin can be present in any suitable concentration. In some embodiments, the concentration of sodium saccharin ranges from 10 ppm to 10000 ppm, or from 50 ppm to 5000 ppm, or from 100 ppm to 3000 ppm.

In some embodiments, the sweetener may comprise caloric sugar, such as sucrose, glucose, fructose (e.g., in the form of high-fructose corn syrup), or any combination thereof. In some embodiments, the sweetener comprises one or more high-intensity artificial sweeteners, such as acefulfame potassium, aspartame, cyclamate, neotame, and the like. When any such artificial high-intensity sweeteners are present, their concentration would be about the same as those set forth above for sucralose. In some other embodiments, the sweetener comprises one or more low-calorie carbohydrates or sugar alcohols, such as allulose, xylitol, erythritol, and the like.

Various other sweeteners may also be included in the flavored product of the present disclosure. Non-limiting examples include D-psicose, L-ribose, D-tagatose, L-glucose, L-fucose, L-arbinose, D-turanose, D-leucrose, isomalt, lactitol, mannitol, sorbitol, maltodextrin, saccharin, alitame, cyclamic acid, tagatose, maltose, galactose, mannose, lactose, D-tryptophan, glycine, maltitol, lactitol, isomalt, hydrogenated starch hydrolyzate (HSH), chemically modified mogrosides (such as glucosylated mogrosides), carrelame and other guanidine-based sweeteners, honey, Jerusalem artichoke syrup, licorice root, luo han guo (fruit, powder, or extracts), lucuma (fruit, powder, or extracts), maple sap (including, for example, sap extracted from Acer saccharum, Acer nigrum, Acer rubrum, Acer saccharinum, Acer platanoides, Acer negundo, Acer macrophyllum, Acer grandidentatum, Acer glabrum, Acer mono), maple syrup, maple sugar, walnut sap (including, for example, sap extracted from Juglans cinerea, Juglans nigra, Juglans ailatifolia, Juglans regia), birch sap (including, for example, sap extracted from Betula papyrifera, Betula alleghaniensis, Betula lenta, Betula nigra, Betula populifolia, Betula pendula), sycamore sap (such as, for example, sap extracted from Platanus occidentalis), ironwood sap (such as, for example, sap extracted from Ostrya virginiana), mascobado, molasses (such as, for example, blackstrap molasses), molasses sugar, monatin, monellin, cane sugar (also referred to as natural sugar, unrefined cane sugar, or sucrose), palm sugar, panocha, piloncillo, rapadura, raw sugar, rice syrup, sorghum, sorghum syrup, cassava syrup (also referred to as tapioca syrup), thaumatin, yacon root, malt syrup, barley malt syrup, barley malt powder, beet sugar, cane sugar, crystalline juice crystals, caramel, carbitol, carob syrup, castor sugar, hydrogenated starch hydrolates, hydrolyzed can juice, hydrolyzed starch, invert sugar, anethole, arabinogalactan, arrope, syrup, P-4000, acesulfame potassium (also referred to as acesulfame K or ace-K), alitame (also referred to as aclame), advantame, aspartame, baiyunoside, neotame, benzamide derivatives, bernadame, canderel, carrelame and other guanidine-based sweeteners, vegetable fiber, corn sugar, coupling sugars, curculin, cyclamates, cyclocarioside I, demerara, dextran, dextrin, diastatic malt, dulcin, sucrol, valzin, dulcoside A, dulcoside B, emulin, enoxolone, maltodextrin, saccharin, estragole, ethyl maltol, glucin, gluconic acid, glucono-lactone, glucosamine, glucoronic acid, glycerol, glycine, glycyphillin, glycyrrhizin, glycyrrhetic acid monoglucuronide, golden sugar, yellow sugar, golden syrup, granulated sugar, gynostemma, hernandulcin, isomerized liquid sugars, jallab, chicory root dietary fiber, kynurenine derivatives (including N′-formyl-kynurenine, N′-acetyl-kynurenine, 6-chloro-kynurenine), galactitol, litesse, ligicane, lycasin, lugduname, guanidine, falernum, mabinlin I, mabinlin II, maltol, maltisorb, maltodextrin, maltotriol, mannosamine, miraculin, mizuame, mukurozioside, nano sugar, naringin dihydrochalcone, neohesperidine dihydrochalcone, nib sugar, nigero-oligosaccharide, norbu, orgeat syrup, osladin, pekmez, pentadin, periandrin I, perillaldehyde, perillartine, petphyllum, phenylalanine, phlomisoside I, phlorodizin, phyllodulcin, polyglycitol syrups, polypodoside A, pterocaryoside A, pterocaryoside B, rebiana, refiners syrup, rub syrup, rubusoside, selligueain A, shugr, siamenoside I, siraitia grosvenorii, soybean oligosaccharide, Splenda, SRI oxime V, steviol glycoside, steviolbioside, stevioside, strogins 1, 2, and 4, sucronic acid, sucrononate, sugar, suosan, phloridzin, superaspartame, tetrasaccharide, threitol, treacle, trilobtain, tryptophan and derivatives (6-trifluoromethyl-tryptophan, 6-chloro-D-tryptophan), vanilla sugar, volemitol, birch syrup, aspartame-acesulfame, assugrin, and combinations or blends of any two or more thereof.

In some embodiments, the flavored product may comprise 4-amino-5,6-dimethylthieno[2,3-d]pyrimidin-2 (1H)-one, or any suitable salt thereof. In some embodiments, the flavored product disclosed herein may comprise the hydrochloride salt of 4-amino-5,6-dimethylthieno[2,3-d]pyrimidin-2 (1H)-one.

In some embodiments of any of the foregoing embodiments, the flavored product may comprise a lactone. Any suitable lactone or combination of lactones can be used. In some embodiments, the lactone comprises a lactone ring having from 3 to 8 members in the ring, such as a 3-membered lactone ring, a 4-membered lactone ring, a 5-membered lactone ring, a 6-membered lactone ring, a 7-membered lactone ring, or an 8-membered lactone ring.

In some embodiments, the lactone comprises a 5-membered lactone ring with or without an unsaturated bond (excluding the unsaturation in the oxo substituent on the ring), such as a carbon-carbon double bond. In some embodiments, the lactone comprises a 5-membered lactone ring including at least one unsaturated bond (for example, one carbon-carbon double bond). Non-limiting examples of lactones comprising a 5-membered lactone ring having at least one unsaturated bond include angelica lactone alpha, angelica lactone beta, mint lactone, 5,5-dimethyl-2 (5H)-furanone, 5-ethyl-2 (5H)-furanone, 5-pentyl-2 (5H)-furanone, 5-hexyl-2 (5H)-furanone, 5-pentyl-2 (3H)-furanone, 4-methyl-3-pentyl-2 (5H)-furanone, and 2 (5H)-furanone. In some embodiments, the lactone comprises a 5-membered lactone ring without any unsaturated bonds. Non-limiting examples of such lactones include butyrolactone gamma, valerolactone gamma, hexalactone gamma, heptalactone gamma, octalactone gamma, undecalactone gamma, decalactone gamma, dihydrojasmone lactone, and dihydromintlactone (3,6-dimethyl-hexahydro-2 (3H)-benzofuranone).

In some embodiments, the lactone comprises a 6-membered lactone ring with or without an unsaturated bond. In some embodiments, the lactone comprises a 6-membered lactone ring including at least one unsaturated bond. Non-limiting examples of lactones comprising a 6-membered lactone ring having at least one unsaturated bond include δ-2-decenolactone, coumarin, and 6-methylcoumarin. In some other embodiments, the lactone includes a 6-membered lactone ring without any unsaturated bonds. Non-limiting examples of lactones including a 6-membered lactone ring without any unsaturated bonds include decalactone delta, dodecalactone delta, and cyclohexyl lactone.

In certain embodiments, the flavored product further comprises one or more food additives, including, but not limited to:
acids, including, for example citric acid, phosphoric acid, ascorbic acid, sodium acid sulfate, lactic acid, or tartaric acid;
bitter ingredients, including, for example caffeine, quinine, green tea, catechins, polyphenols, green robusta coffee extract, green coffee extract, potassium chloride, menthol, or proteins (such as proteins and protein isolates derived from plants, algae, or fungi);
coloring agents, including, for example caramel color, Red #40, Yellow #5, Yellow #6, Blue #1, Red #3, purple carrot, black carrot juice, purple sweet potato, vegetable juice, fruit juice, beta carotene, turmeric curcumin, or titanium dioxide;
preservatives, including, for example sodium benzoate, potassium benzoate, potassium sorbate, sodium metabisulfate, sorbic acid, or benzoic acid;
antioxidants including, for example ascorbic acid, calcium disodium EDTA, alpha tocopherols, mixed tocopherols, rosemary extract, grape seed extract, resveratrol, or sodium hexametaphosphate;
vitamins or functional ingredients including, for example resveratrol, Co-Q10, omega 3 fatty acids, theanine, choline chloride (citocoline), fibersol, inulin (chicory root), taurine, panax ginseng extract, guanana extract, ginger extract, L-phenylalanine, L-carnitine, L-tartrate, D-glucoronolactone, inositol, bioflavonoids, Echinacea, ginko biloba, yerba mate, flax seed oil, garcinia cambogia rind extract, white tea extract, ribose, milk thistle extract, grape seed extract, pyrodixine HCl (vitamin B6), cyanoobalamin (vitamin B12), niacinamide (vitamin B3), biotin, calcium lactate, calcium pantothenate (pantothenic acid), calcium phosphate, calcium carbonate, chromium chloride, chromium polynicotinate, cupric sulfate, folic acid, ferric pyrophosphate, iron, magnesium lactate, magnesium carbonate, magnesium sulfate, monopotassium phosphate, monosodium phosphate, phosphorus, potassium iodide, potassium phosphate, riboflavin, sodium sulfate, sodium gluconate, sodium polyphosphate, sodium bicarbonate, thiamine mononitrate, vitamin D3, vitamin A palmitate, zinc gluconate, zinc lactate, or zinc sulphate;
clouding agents, including, for example ester gun, brominated vegetable oil (BVO), or sucrose acetate isobutyrate (SAIB);
buffers, including, for example sodium citrate, potassium citrate, or salt;
flavors, including, for example natural flavor, natural flavor with other natural flavors (natural flavor WONF), natural and artificial flavors, artificial flavor; or
starches and stabilizers, including, for example pectin, xanthan gum, carboxylmethylcellulose (CMC), polysorbate 60, polysorbate 80, medium chain triglycerides, cellulose gel, cellulose gum, sodium caseinate, modified food starch, gum Arabic (gum acacia), inulin, or carrageenan.

In some further embodiments, the flavored product disclosed herein comprises one or more other umami or kokumi enhancing compounds. Such umami enhancing compounds include, but are not limited to, naturally derived compounds, such as ericamide, or synthetic compounds, such as any compounds set forth in U.S. Pat. Nos. 8,735,081; 8,124,121; and 8,968,708.

In some further embodiments, the flavored product disclosed herein may comprise one or more bitterness blocking compounds. Such bitterness blocking compounds include, but are not limited to, naturally derived compounds, such as menthol or analogs thereof, or synthetic compounds, such as any compounds set forth in U.S. Pat. Nos. 8,076,491; 8,445,692; and 9,247,759.

In some further embodiments, the flavored product disclosed herein may comprise one or more mouthfeel modifying compounds. Such mouthfeel modifying compounds include, but are not limited to, tannins, cellulosic materials, bamboo powder, and the like.

In some further embodiments, the flavored product disclosed herein may comprise one or more flavor masking compounds. Such flavor masking compounds include, but are not limited to, cellulosic materials, materials extracted from fungus, materials extracted from plants, citric acid, carbonic acid (or carbonates), and the like.

In certain embodiments, the flavored product disclosed herein may comprise cyclosal. When cyclosal is present, it is present at a relative concentration similar to that of the lactones described herein.

In some embodiments, the flavored product comprises one or more flavorings, or any other additives described in H. Mitchell, Sweeteners and Sugar Alternatives in Food Technology, Blackwell Publishing Ltd, 2006. Useful flavorings are artificial, natural and synthetic fruit flavors, such as vanilla. Other potential flavors include a milk flavor, a butter flavor, a cheese flavor, a cream flavor, and a yogurt flavor; a vanilla flavor; tea or coffee flavors, such as a green tea flavor, a oolong tea flavor, a tea flavor, a cocoa flavor, a chocolate flavor, and a coffee flavor; mint flavors, such as a peppermint flavor, a spearmint flavor, and a Japanese mint flavor; spicy flavors, such as an asafetida flavor, an ajowan flavor, an anise flavor, an angelica flavor, a fennel flavor, an allspice flavor, a cinnamon flavor, a camomile flavor, a mustard flavor, a cardamom flavor, a caraway flavor, a cumin flavor, a clove flavor, a pepper flavor, a coriander flavor, a sassafras flavor, a savory flavor, a zanthoxyli Fructus flavor, a perilla flavor, a juniper berry flavor, a ginger flavor, a star anise flavor, a horseradish flavor, a thyme flavor, a tarragon flavor, a dill flavor, a capsicum flavor, a nutmeg flavor, a basil flavor, a marjoram flavor, a rosemary flavor, a bayleaf flavor, and a wasabi (Japanese horseradish) flavor; alcoholic flavors, such as a wine flavor, a whisky flavor, a brandy flavor, a rum flavor, a gin flavor, and a liqueur flavor; floral flavors; and vegetable flavors, such as an onion flavor, a garlic flavor, a cabbage flavor, a carrot flavor, a celery flavor, mushroom flavor, and a tomato flavor. These flavoring agents may be used in liquid or solid form and may be used individually or in admixture.

Flavors may also provide breath freshening properties, particularly the mint flavors when used in combination with cooling agents. These flavorings may be used in liquid or solid form and may be used individually or in admixture. Other useful flavorings include aldehydes and esters such as cinnamyl acetate, cinnamaldehyde, citral diethylacetal, dihydrocarvyl acetate, eugenyl formate, p-methylamisol, and so forth may be used. Generally, any flavoring or food additive such as those described in Chemicals Used in Food Processing, publication 1274, pages 63-258, by the National Academy of Sciences, may be used.

Further examples of aldehyde flavorings include but are not limited to acetaldehyde (apple), benzaldehyde (cherry, almond), anisic aldehyde (licorice, anise), cinnamic aldehyde (cinnamon), citral, i.e., alpha-citral (lemon, lime), neral, i.e., beta-citral (lemon, lime), decanal (orange, lemon), ethyl vanillin (vanilla, cream), heliotrope, i.e., piperonal (vanilla, cream), vanillin (vanilla, cream), alpha-amyl cinnamaldehyde (spicy fruity flavors), butyraldehyde (butter, cheese), valeraldehyde (butter, cheese), citronellal (modifies, many types), decanal (citrus fruits), aldehyde C-8 (citrus fruits), aldehyde C-9 (citrus fruits), aldehyde C-12 (citrus fruits), 2-ethyl butyraldehyde (berry fruits), hexenal, i.e., trans-2 (berry fruits), tolyl aldehyde (cherry, almond), veratraldehyde (vanilla), 2,6-dimethyl-5-heptenal, i.e., melonal (melon), 2,6-dimethyloctanal (green fruit), and 2-dodecenal (citrus, mandarin), cherry, grape, strawberry shortcake, and mixtures thereof. These listings of flavorings are merely exemplary and are not meant to limit either the term “flavoring” or the scope of the disclosure generally.

The flavored product can be any flavored product according to the aspects and embodiments set forth below. In some embodiments, the flavored product is a beverage product, such as enhanced sparkling beverages, colas, lemon-lime flavored sparkling beverages, orange flavored sparkling beverages, grape flavored sparkling beverages, strawberry flavored sparkling beverages, pineapple flavored sparkling beverages, ginger-ales, root beers, fruit juices, fruit-flavored juices, juice drinks, nectars, vegetable juices, vegetable-flavored juices, sports drinks, energy drinks, enhanced water drinks, enhanced water with vitamins, near water drinks, coconut waters, tea type drinks, coffees, cocoa drinks, beverages containing milk components, beverages containing cereal extracts and smoothies. In some other embodiments, the flavored product is a food product, such as a packaged meal, canned foods, meal replacement products, and the like. In some embodiments, the flavored product is an oral care product, such as a toothpaste, a mouthwash, or a teeth-whitening composition, such as a composition disclosed in U.S. Patent Application Pub. No. 2019/0167545, which is incorporated herein by reference.

In embodiments where the flavored product is a beverage, the beverage may be selected from the group consisting of enhanced sparkling beverages, colas, lemon-lime flavored sparkling beverages, orange flavored sparkling beverages, grape flavored sparkling beverages, strawberry flavored sparkling beverages, pineapple flavored sparkling beverages, ginger-ales, root beers, fruit juices, fruit-flavored juices, juice drinks, nectars, vegetable juices, vegetable-flavored juices, sports drinks, energy drinks, enhanced water drinks, enhanced water with vitamins, near water drinks, coconut waters, tea type drinks, coffees, cocoa drinks, beverages containing milk components, beverages containing cereal extracts and smoothies. In some embodiments, the beverage may be a soft drink.

The flavored product can be in any suitable physical form. For example, in some embodiments, the flavored product can be in the form of a solid, such as a powder. In a further such embodiment, the flavored product is a toothpowder, such as a toothpowder for whitening teeth, removing stains (e.g., tobacco stains), and the like. In some other embodiments, the flavored product can be a paste or a slurry, such as a toothpaste. In some other embodiments, the flavored product can be in the form of a cream or gel, such as a tooth gel, a skin cream or tooth cream. In some other embodiments, the flavored product can be a liquid suspension or solution, for example, with an aqueous carrier. The flavored product can, in some embodiments, be in the form of an emulsion, such as a microemulsion or nanoemulsion, where a surfactant, emulsifier, and the like can be present. In some embodiments, the flavored product is a personal care, such as a product for application onto skin, face, lips, gums, mouth, and the like. Such personal products can be for any suitable use, such as skin whitening, teeth whitening, and the like.

In some embodiments, the flavored product is an oral care product. By “oral care product” is meant a personal care product or other product, which in the ordinary course of usage, is not intentionally swallowed for purposes of systemic administration of particular therapeutic agents, but is rather retained in the oral cavity for a time sufficient to contact substantially all of the dental surfaces and/or oral tissues for purposes of oral activity. The oral care composition may be in various forms including toothpaste, toothpaste powder, dentifrice, tooth gel, subgingival gel, mouthrinse, mousse, foam, mouthspray, lozenge, chewable tablet, chewing gum or denture product. The oral care composition may also be incorporated onto strips or films for direct application or attachment to oral surfaces.

In some embodiments, the flavored product is a pharmaceutical product, such as a product designed to carry or deliver certain active pharmaceutical ingredients (APIs). Such APIs can be over-the-counter (OTC) drugs, such as acetaminophen and other OTC cough and cold medications. In some embodiments, the API is a prescription-based drug.

In certain embodiments of any aspects and embodiments set forth herein that refer to a flavored product, the flavored product is a non-naturally-occurring product, such as a packaged food or beverage product.

Further non-limiting examples of food and beverage products or formulations include sweet coatings, frostings, or glazes for such products or any entity included in the Soup category, the Dried Processed Food category, the Beverage category, the Ready Meal category, the Canned or Preserved Food category, the Frozen Processed Food category, the Chilled Processed Food category, the Snack Food category, the Baked Goods category, the Confectionery category, the Dairy Product category, the Ice Cream category, the Meal Replacement category, the Pasta and Noodle category, and the Sauces, Dressings, Condiments category, the Baby Food category, and/or the Spreads category.

In general, the Soup category refers to canned/preserved, dehydrated, instant, chilled, UHT and frozen soup. For the purpose of this definition soup(s) means a food prepared from meat, poultry, fish, vegetables, grains, fruit and other ingredients, cooked in a liquid which may include visible pieces of some or all of these ingredients. It may be clear (as a broth) or thick (as a chowder), smooth, pureed or chunky, ready-to-serve, semi-condensed or condensed and may be served hot or cold, as a first course or as the main course of a meal or as a between meal snack (sipped like a beverage). Soup may be used as an ingredient for preparing other meal components and may range from broths (consommé) to sauces (cream or cheese-based soups).

The Dehydrated and Culinary Food Category usually means: (i) Cooking aid products such as: powders, granules, pastes, concentrated liquid products, including concentrated bouillon, bouillon and bouillon like products in pressed cubes, tablets or powder or granulated form, which are sold separately as a finished product or as an ingredient within a product, sauces and recipe mixes (regardless of technology); (ii) Meal solutions products such as: dehydrated and freeze dried soups, including dehydrated soup mixes, dehydrated instant soups, dehydrated ready-to-cook soups, dehydrated or ambient preparations of ready-made dishes, meals and single serve entrees including pasta, potato and rice dishes; and (iii) Meal embellishment products such as: condiments, marinades, salad dressings, salad toppings, dips, breading, batter mixes, shelf stable spreads, barbecue sauces, liquid recipe mixes, concentrates, sauces or sauce mixes, including recipe mixes for salad, sold as a finished product or as an ingredient within a product, whether dehydrated, liquid or frozen.

The Beverage category usually means beverages, beverage mixes and concentrates, including but not limited to, carbonated and non-carbonated beverages, alcoholic and non-alcoholic beverages, ready to drink beverages, liquid concentrate formulations for preparing beverages such as sodas, and dry powdered beverage precursor mixes. The Beverage category also includes the alcoholic drinks, the soft drinks, sports drinks, isotonic beverages, and hot drinks. The alcoholic drinks include, but are not limited to beer, cider/perry, FABs, wine, and spirits. The soft drinks include, but are not limited to carbonates, such as colas and non-cola carbonates; fruit juice, such as juice, nectars, juice drinks and fruit flavored drinks; bottled water, which includes sparkling water, spring water and purified/table water; functional drinks, which can be carbonated or still and include sport, energy or elixir drinks; concentrates, such as liquid and powder concentrates in ready to drink measure. The drinks, either hot or cold, include, but are not limited to coffee or ice coffee, such as fresh, instant, and combined coffee; tea or iced tea, such as black, green, white, oolong, and flavored tea; and other drinks including flavor-, malt- or plant-based powders, granules, blocks or tablets mixed with milk or water.

The Snack Food category generally refers to any food that can be a light informal meal including, but not limited to Sweet and savory snacks and snack bars. Examples of snack food include, but are not limited to fruit snacks, chips/crisps, extruded snacks, tortilla/corn chips, popcorn, pretzels, nuts and other sweet and savory snacks. Examples of snack bars include, but are not limited to granola/muesli bars, breakfast bars, energy bars, fruit bars and other snack bars.

The Baked Goods category generally refers to any edible product the process of preparing which involves exposure to heat or excessive sunlight. Examples of baked goods include, but are not limited to bread, buns, cookies, muffins, cereal, toaster pastries, pastries, waffles, tortillas, biscuits, pies, bagels, tarts, quiches, cake, any baked foods, and any combination thereof. Further examples include, but are not limited to packaged/industrial bread, unpackaged/artisanal bread, packaged/industrial cakes, unpackaged/artisanal cakes, cookies, chocolate coated biscuits, sandwich biscuits, filled biscuits, savory biscuits and crackers, bread substitutes, breakfast cereals, rte cereals, family breakfast cereals, flakes, muesli, other cereals, children's breakfast cereals, and hot cereals.

The Ice Cream category generally refers to frozen dessert containing cream and sugar and flavoring. Examples of ice cream include, but are not limited to, impulse ice cream; take-home ice cream; frozen yoghurt and artisanal ice cream; soy, oat, bean (e.g., red bean and mung bean), and rice-based ice creams. Further examples of ice cream include, but are not limited to, single portion dairy ice cream, single portion water ice cream, multi-pack dairy ice cream, multi-pack water ice cream, take-home dairy ice cream, ice cream desserts, bulk ice cream, and take-home water ice cream.

The Confectionery category generally refers to edible product that is sweet to the taste. Examples of confectionery include, but are not limited to candies, gelatins, chocolate confectionery, sugar confectionery, gum, and the likes and any combination products. Further examples include, but are not limited to, tablets, countlines, bagged selflines/softlines, boxed assortments, standard boxed assortments, twist wrapped miniatures, seasonal chocolate, chocolate with toys, alfajores, other chocolate confectionery, mints, standard mints, power mints, boiled sweets, pastilles, gums, jellies and chews, toffees, caramels and nougat, medicated confectionery, lollipops, liquorice, and other sugar confectionery.

The Meal Replacement category generally refers to any food intended to replace the normal meals, particularly for people having health or fitness concerns. Examples of meal replacement include, but are not limited to, slimming products and convalescence drinks, and convalescence products.

The Ready Meal category generally refers to any food that can be served as meal without extensive preparation or processing. The ready meal includes products that have had recipe “skills” added to them by the manufacturer, resulting in a high degree of readiness, completion and convenience. Examples of ready meal include, but are not limited to canned/preserved, frozen, dried, chilled ready meals; dinner mixes; frozen pizza; chilled pizza; and prepared salads.

The Pasta and Noodle category includes any pastas and/or noodles including, but not limited to canned, dried and chilled/fresh pasta; and plain, instant, chilled, frozen and snack noodles.

The Canned/Preserved Food category includes, but is not limited to, canned/preserved meat and meat products, fish/seafood, vegetables, tomatoes, beans, fruit, ready meals, soup, pasta, and other canned/preserved foods.

The Frozen Processed Food category includes, but is not limited to frozen processed red meat, processed poultry, processed fish/seafood, processed vegetables, meat substitutes, processed potatoes, bakery products, desserts, ready meals, pizza, soup, noodles, and other frozen food.

The Dried Processed Food category includes, but is not limited to rice, dessert mixes, dried ready meals, dehydrated soup, instant soup, dried pasta, plain noodles, and instant noodles. The Chill Processed Food category includes, but is not limited to chilled processed meats, processed fish/seafood products, lunch kits, fresh cut fruits, ready meals, pizza, prepared salads, soup, fresh pasta and noodles.

The Sauces, Dressings and Condiments category includes, but is not limited to tomato pastes and purees, bouillon/stock cubes, herbs and spices, monosodium glutamate (MSG), table sauces, soy-based sauces, pasta sauces, wet/cooking sauces, dry sauces/powder mixes, ketchup, mayonnaise, mustard, salad dressings, vinaigrettes, dips, pickled products, and other sauces, dressings and condiments.

The Baby Food category includes, but is not limited to, milk- or soybean-based formula; and prepared, dried and other baby food.

The Spreads category includes, but is not limited to jams and preserves, honey, chocolate spreads, nut-based spreads, and yeast-based spreads.

The Dairy Product category generally refers to edible product produced from mammal's milk. Examples of dairy products include, but are not limited to drinking milk products, cheese, yoghurt and sour milk drinks, and other dairy products.

Further examples of dairy products include, but are not limited to, fresh/pasteurized milk, full fat fresh/pasteurized milk, semi skimmed fresh/pasteurized milk, long-life/uht milk, full fat long life/uht milk, semi skimmed long life/uht milk, fat-free long life/uht milk, goat milk, condensed/evaporated milk, plain condensed/evaporated milk, flavored, functional and other condensed milk, flavored milk drinks, dairy only flavored milk drinks, flavored milk drinks with fruit juice, soy milk, fermented dairy drinks, coffee whiteners, powder milk, flavored powder milk drinks, cream, processed cheese, spreadable processed cheese, unspreadable processed cheese, unprocessed cheese, spreadable unprocessed cheese, hard cheese, packaged hard cheese, unpackaged hard cheese, plain/natural yoghurt, flavored yoghurt, fruited yoghurt, probiotic yoghurt, drinking yoghurt, regular drinking yoghurt, and probiotic drinking yoghurt.

In some embodiments, the flavored product comprises one or more confectioneries, chocolate confectionery, tablets, countlines, bagged selflines/softlines, boxed assortments, standard boxed assortments, twist wrapped miniatures, seasonal chocolate, chocolate with toys, alfajores, other chocolate confectionery, mints, standard mints, power mints, boiled sweets, pastilles, gums, jellies and chews, toffees, caramels and nougat, medicated confectionery, lollipops, liquorice, other sugar confectionery, bread, packaged/industrial bread, unpackaged/artisanal bread, pastries, cakes, packaged/industrial cakes, unpackaged/artisanal cakes, cookies, chocolate coated biscuits, sandwich biscuits, filled biscuits, savory biscuits and crackers, bread substitutes, breakfast cereals, rte cereals, family breakfast cereals, flakes, muesli, other cereals, children's breakfast cereals, hot cereals, ice cream, impulse ice cream, single portion dairy ice cream, single portion water ice cream, multi-pack dairy ice cream, multi-pack water ice cream, take-home ice cream, take-home dairy ice cream, ice cream desserts, bulk ice cream, take-home water ice cream, frozen yoghurt, artisanal ice cream, dairy products, milk, fresh/pasteurized milk, full fat fresh/pasteurized milk, semi skimmed fresh/pasteurized milk, long-life/uht milk, full fat long life/uht milk, semi skimmed long life/uht milk, fat-free long life/uht milk, goat milk, condensed/evaporated milk, plain condensed/evaporated milk, flavored, functional and other condensed milk, flavored milk drinks, dairy only flavored milk drinks, flavored milk drinks with fruit juice, soy milk, sour milk drinks, fermented dairy drinks, coffee whiteners, powder milk, flavored powder milk drinks, cream, cheese, processed cheese, spreadable processed cheese, unspreadable processed cheese, unprocessed cheese, spreadable unprocessed cheese, hard cheese, packaged hard cheese, unpackaged hard cheese, yoghurt, plain/natural yoghurt, flavored yoghurt, fruited yoghurt, probiotic yoghurt, drinking yoghurt, regular drinking yoghurt, probiotic drinking yoghurt, chilled and shelf-stable desserts, dairy-based desserts, soy-based desserts, chilled snacks, fromage frais and quark, plain fromage frais and quark, flavored fromage frais and quark, savory fromage frais and quark, sweet and savory snacks, fruit snacks, chips/crisps, extruded snacks, tortilla/corn chips, popcorn, pretzels, nuts, other sweet and savory snacks, snack bars, granola bars, breakfast bars, energy bars, fruit bars, other snack bars, meal replacement products, slimming products, convalescence drinks, ready meals, canned ready meals, frozen ready meals, dried ready meals, chilled ready meals, dinner mixes, frozen pizza, chilled pizza, soup, canned soup, dehydrated soup, instant soup, chilled soup, hot soup, frozen soup, pasta, canned pasta, dried pasta, chilled/fresh pasta, noodles, plain noodles, instant noodles, cups/bowl instant noodles, pouch instant noodles, chilled noodles, snack noodles, canned food, canned meat and meat products, canned fish/seafood, canned vegetables, canned tomatoes, canned beans, canned fruit, canned ready meals, canned soup, canned pasta, other canned foods, frozen food, frozen processed red meat, frozen processed poultry, frozen processed fish/seafood, frozen processed vegetables, frozen meat substitutes, frozen potatoes, oven baked potato chips, other oven baked potato products, non-oven frozen potatoes, frozen bakery products, frozen desserts, frozen ready meals, frozen pizza, frozen soup, frozen noodles, other frozen food, dried food, dessert mixes, dried ready meals, dehydrated soup, instant soup, dried pasta, plain noodles, instant noodles, cups/bowl instant noodles, pouch instant noodles, chilled food, chilled processed meats, chilled fish/seafood products, chilled processed fish, chilled coated fish, chilled smoked fish, chilled lunch kit, chilled ready meals, chilled pizza, chilled soup, chilled/fresh pasta, chilled noodles, oils and fats, olive oil, vegetable and seed oil, cooking fats, butter, margarine, spreadable oils and fats, functional spreadable oils and fats, sauces, dressings and condiments, tomato pastes and purees, bouillon/stock cubes, stock cubes, gravy granules, liquid stocks and fonds, herbs and spices, fermented sauces, soy based sauces, pasta sauces, wet sauces, dry sauces/powder mixes, ketchup, mayonnaise, regular mayonnaise, mustard, salad dressings, regular salad dressings, low fat salad dressings, vinaigrettes, dips, pickled products, other sauces, dressings and condiments, baby food, milk formula, standard milk formula, follow-on milk formula, toddler milk formula, hypoallergenic milk formula, prepared baby food, dried baby food, other baby food, spreads, jams and preserves, honey, chocolate spreads, nut-based spreads, and yeast-based spreads.

Some embodiments provide a chewable flavored product that may or may not be intended to be swallowed. In some embodiments, the chewable flavored product may be gum, chewing gum, sugarized gum, sugar-free gum, functional gum, bubble gum including compounds as disclosed and described herein, individually or in combination.

The flavored products disclosed herein contain one or more compounds that users of those products perceive as having an undesirable taste, such as a bitter taste, a medicinal taste, or a metallic taste. As used herein, such compounds are referred to as unpleasant tastants. In some embodiments, the flavored product comprises an unpleasant tastant, wherein unpleasant tastant is cetylpyridinium chloride, lauramidopropyl betaine, cocamidopropyl betaine, arginine, hydrogen peroxide, chlorhexidine gluconate, zinc phosphate, zinc chloride, zinc citrate, potassium nitrate, pentasodium triphosphate, tetrasodium pyrophosphate, stannous fluoride, sodium bicarbonate, thymol, menthol, methyl salicylate, eucalyptol, or any combination thereof. In some further embodiments, the unpleasant tastant is thymol, menthol, methyl salicylate, eucalyptol, or any combination thereof. In some embodiments, the unpleasant tastant is cetylpyridinium chloride, lauramidopropyl betaine, cocamidopropyl betaine, or any combination thereof. In some embodiments, the unpleasant tastant is arginine. In some embodiments, the unpleasant tastant is hydrogen peroxide. In some embodiments, the unpleasant tastant is chlorhexidine gluconate. In some embodiments, the unpleasant tastant is zinc phosphate, zinc chloride, zinc citrate, potassium nitrate, pentasodium triphosphate, tetrasodium pyrophosphate, stannous fluoride, or any combinations thereof. In some embodiments, the unpleasant tastant is sodium bicarbonate.

The unpleasant tastant can be present in the flavored product in any suitable amount. For example, in embodiments where the flavored product is an oral care product, the unpleasant tastant(s) can be present in any amount that is typical for use with oral care products of similar type.

In the third aspect, the present disclosure relates to the use of the taste-modifying composition described herein to reduce an unpleasant taste of a flavored product. The unpleasant taste is often attributed to the presence of one or more compounds (unpleasant tastant) that users of those products perceive as having an undesirable taste, such as a bitter taste, a medicinal taste, or a metallic taste. Mention is also made of a method for reducing an unpleasant taste of a flavored product having one or more unpleasant tastants, the method comprising adding the taste-modifying composition to the flavored product.

The compositions, products, and uses according to the present disclosure are further illustrated by the following non-limiting examples.

### Example 1. Taste-Modifying Compositions

Taste-modifying compositions according to some aspects of the present disclosure were prepared and summarized in Tables 1a and 1b below. Unless otherwise indicated, the amount of each ingredient is provided in weight percent relative to the total weight of the composition.

TABLE 1a

| | Compo- | Compo- | Compo- | Compo- |
|---|---|---|---|---|
| Ingredient | sition 1 | sition 2 | sition 3 | sition 4 |
| Peppermint flavor oil | 43.1 | 44.1 | 45.3 | 49.4 |
| GSG | 2.2 | 0.44 | 0.45 | 0.49 |
| Span 20 | 28.0 | 28.7 | 7.1 | 6.2 |
| Poloxamer 407 | 15.1 | 15.4 | 15.7 | 24.7 |
| Propylene glycol | 3.0 | 3.0 | | |
| Ethyl lactate | | | 22.8 | 9.8 |
| Water | 8.6 | 8.36 | 8.65 | 9.41 |
| Ratio of peppermint | 20 | 100 | 100 | 100 |
| flavor oil/GSG | | | | |

TABLE 1b

| | Composition | Composition | Composition |
|---|---|---|---|
| Ingredient | 5 | 6 | 7 |
| Peppermint flavor oil | 41.8 | 42.1 | 44.7 |
| GSG | 0.42 | 0.42 | 0.45 |
| Span 20 | 20.0 | 15.4 | 17.3 |
| Poloxamer 407 | 18.3 | 9.6 | 14.2 |
| Propylene glycol | 0.4 | 8.4 | 13.7 |
| Ethyl lactate | 10.9 | 21.1 | 2.3 |
| Water | 8.3 | 3.0 | 7.4 |
| | 41.8 | 42.1 | 44.7 |
| Ratio of peppermint flavor | 100 | 100 | 100 |
| oil/GSG | | | |

Samples 1-7 were prepared as follows:

Preparation of oil phase: the flavor oil, non-polar and polar surfactants, and the water-miscible solvent were mixed and heated under gentle stirring (45° C.) and until a homogenous liquid is obtained.

Preparation of aqueous phase: GSG was mixed with water and stirred at RT until complete dissolution of the GSG.

Preparation of the water-in-oil microemulsion: the oil phase and the aqueous phase were mixed and stirred at RT until a homogenous liquid is obtained.

The resulting compositions were transparent at room temperature (RT) and remained stable for at least three months.

### Example 2. Sensory Evaluation

Samples 3 and 4 were evaluated in a sensory test. Two comparative toothpastes and two toothpastes according to some aspects of the present disclosure were produced using the toothpaste base shown in Table 2. Two comparative toothpastes and two toothpastes according to some aspects of the present disclosure are summarized in Table 3.

TABLE 2
Composition of the toothpaste base

| | Concentration |
|---|---|
| Ingredient | (%) |
| Polyethylene glycol 400 | 2.0 |
| Xanthan gum | 0.60 |
| Sorbitol (70% solution in | 50.0 |
| water) | |
| Sodium fluoride | 0.22 |
| Sodium benzoate | 0.20 |
| Hydrated silica (Tixosil 73) | 22.0 |
| Hydrated silica (Tixosil 43) | 7.0 |
| Titanium dioxide | 0.50 |
| Sodium lauryl sulfate | 1.25 |
| Flavor | TBD |
| Sodium saccharin | TBD |
| Water | q.s. to 100% |

TABLE 3
Toothpaste compositions

| | Comparative | Comparative | Inventive | Inventive |
|---|---|---|---|---|
| Ingredient | sample 1 | sample 2 | sample 3 | sample 4 |
| Toothpaste base | 96.0 | 96.0 | 96.0 | 96.0 |
| Saccharin (25% | 0.8 | 0.8 | 0.8 | 0.8 |
| solution in H₂O) | | | | |
| Peppermint flavor | 1.0 | 1.0 | | |
| GSG | | 0.01 | | |
| Composition 3 | | | 2.0 | |
| Composition 4 | | | | 2.0 |
| Water | 2.2 | 2.19 | 1.2 | 1.2 |

Sensory testers (panel of 4 experts) were asked to perform the following: (a) apply approximately 1-2 g of sample on the toothbrush, (b) rinse the toothbrush under running water for 5 seconds to moisten the paste and soften the bristles, (c) brush around the teeth for 1 minute, minimizing brushing of gums, and (d) evaluate off-note intensity while brushing and 3 minutes after brushing using a 0-to-5 intensity scale (0 indicating no intensity and 5 indicating very strong intensity). The results are presented in Table 4.

TABLE 4
WHILE BRUSHING
Off-note intensity (0-not at all, 5-Very strong)

| | | | | |
|---|---|---|---|---|
| Bitterness | 4.5 | 3.5 | 3 | 2 |
| Astringency | 4 | 4 | 5.5 | 3.5 |
| Burning | 1.5 | 1 | 1 | 1 |

3 MIN AFTER BRUSHING
Off-note intensity (0-not at all, 5-Very strong)

| | | | | |
|---|---|---|---|---|
| Bitterness | 1.5 | 2.5 | 1 | 1 |
| Astringency | 2 | 2.5 | 2.5 | 1.5 |
| Burning | 0.5 | 0.5 | 0.5 | 0.5 |

The disclosed subject matter has been described with reference to specific details of particular embodiments thereof. It is not intended that such details be regarded as limitations upon the scope of the disclosed subject matter except insofar as and to the extent that they are included in the accompanying claims.

Therefore, the exemplary embodiments described herein are well adapted to attain the ends and advantages mentioned as well as those that are inherent therein. The particular embodiments disclosed above are illustrative only, as the exemplary embodiments described herein may be modified and practiced in different but equivalent manners apparent to those of ordinary skill in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope and spirit of the exemplary embodiments described herein. The exemplary embodiments described herein illustratively disclosed herein suitably may be practiced in the absence of any element that is not specifically disclosed herein and/or any optional element disclosed herein.

1. A taste-modifying composition comprising:
a) one or more flavor oils,
b) one or more off-note masking agents,
c) one or more non-polar surfactants,
d) one or more polar surfactants,
e) water, and
f) one or more water-miscible co-solvents.

2. The taste-modifying composition according to claim 1, wherein the one or more flavor oils is/are selected from the group consisting of mint oil; cinnamon oil, oil of wintergreen (methyl salicylate), clove oil, bay oil, anise oil, eucalyptus oil, thyme oil, cedar leaf oil, oil of nutmeg, allspice, oil of sage, mace, oil of bitter almonds, cassia oil, and citrus oil.

3. The taste-modifying composition according to claim 1, wherein the one or more flavor oils is/are present in an amount of from 20% to 60%, by weight relative to the total weight of the composition.

4. The taste-modifying composition according to claim 1, wherein the one or more off-note masking agents comprise(s) one or more glucosylated natural steviol glycosides.

5. The taste-modifying composition according to claim 1, wherein the one or more off-note masking agents is/are present in an amount of from 0.01% to 5%, by weight relative to the total weight of the composition.

6. The taste-modifying composition according to claim 1, wherein the one or more non-polar surfactants each has a hydrophilic-lipophilic balance (HLB) value of equal to or less than 10.

7. The taste-modifying composition according to claim 1, wherein the one or more non-polar surfactants comprise(s) one or more sorbitan fatty acid esters; lecithins; (poly)glyceryl esters; or mixtures thereof.

8. The taste-modifying composition according to claim 1, wherein the one or more non-polar surfactants is/are present in an amount of from 1% to 40%, by weight relative to the total weight of the composition.

9. The taste-modifying composition according to claim 1, wherein the one or more polar surfactants each has a hydrophilic-lipophilic balance (HLB) value of greater than 10.

10. The taste-modifying composition according to claim 1, wherein the one or more polar surfactants is/are selected from the group consisting of poly(oxyethylene)-poly(oxypropylene)-poly(oxyethylene) block copolymers; one or more PEG sorbitan fatty acid esters; PEG-modified hydrogenated castor oil; sugar esters; (poly)glyceryl esters, alkyl glucosides, and mixtures thereof.

11. The taste-modifying composition according to claim 1, wherein the one or more polar surfactants is/are present in an amount of from 1% to 40%, by weight relative to the total weight of the composition.

12. The taste-modifying composition according to claim 1, wherein the one or more water-miscible co-solvents is/are selected from the group consisting of propylene glycol, glycerol, ethanol, triethyl citrate, triacetin, ethyl lactate, ethyl acetate, propanol, isopropanol, and mixtures thereof.

13. The taste-modifying composition according to claim 1, wherein the one or more water-miscible co-solvents is/are present in an amount of from 0.01% to 30%, by weight relative to the total weight of the composition.

14. The taste-modifying composition according to claim 1, wherein the weight ratio of the one or more flavor oils to the one or more off-note masking agents is in the range of from 20:1 to 100:1.

15. The taste-modifying composition according to claim 1, wherein the composition is in the form of a water-in-oil microemulsion.

16. The taste-modifying composition according to claim 15, wherein the water-in-oil microemulsion comprises:
a′) a discontinuous aqueous phase comprising the one or more off-note masking agents and water; and
b′) a continuous oil phase comprising the one or more flavor oils.

17. A flavored product comprising the taste-modifying composition according to claim 1 and an unpleasant tastant.

18. The flavored product according to claim 17, wherein the flavored product is an oral care product.

19. (canceled)

20. The taste-modifying composition according to claim 7, wherein the one or more non-polar surfactants comprise(s) one or more sorbitan fatty acid esters selected from the group consisting of sorbitan monostearate, sorbitan tristearate, sorbitan monolaurate, sorbitan monooleate, sorbitan monopalmitate, and sorbitan trioleate.

21. The taste-modifying composition according to claim 10, wherein the one or more polar surfactants is/are selected from the group consisting of one or more triblock copolymers consisting of a central hydrophobic block of polypropylene glycol flanked by two hydrophilic blocks of polyethylene glycol, PEG sorbitan monolaurate, PEG sorbitan monooleate, PEG sorbitan monopalmitate, PEG sorbitan stearate, sucrose monopalmitate, sucrose monolaurate, decaglyceryl monooleate, and mixtures thereof.
